(19) United States
(12) Patent Application Publication
(10) Pub. No.: US 2009/0305225 A1
(43) Pub. Date: Dec. 10, 2009

(54) Inhibition of Creatine Uptake to Promote Weight Loss

(76) Inventor: Richard A. Galbraith, Shelburne, VT (US)

Correspondence Address: DAVID S. RESNICK, NIXON PEABODY LLP, 100 SUMMER STREET, BOSTON, MA, 02110-2131, US

(21) Appl. No.: 11/922,058

(22) PCT Filed: Jun. 13, 2006
(86) PCT No.: PCT/US2006/022872

§ 371 Date: Jul. 16, 2008

Related U.S. Application Data
(60) Provisional application No. 60/689,820, filed on Jun. 13, 2005.

(51) Int. Cl. A61K 31/195; A61K 31/661; A61P 3/04; G01N 33/53

(52) U.S. Cl. 435/4; 514/565; 514/114

(57) ABSTRACT

The present invention provides a method of promoting weight loss, or treating or preventing a body disorder related to excess weight, in a subject. The method comprises administering to the subject an effective amount of a creatine uptake inhibitor. Administration of the creatine uptake inhibitor is intracranial or directed to the hypothalamus. The invention further provides a method of screening for a novel compound that inhibits creatine uptake in the hypothalamus.

### CROSS REFERENCE TO RELATED APPLICATIONS

This application claims the benefit under 35 U.S.C. § 119(e) of U.S. Provisional Patent Application Ser. No. 60/689,820 filed Jun. 13, 2005, the contents of which are herein incorporated by reference in their entirety.

### GOVERNMENT SUPPORT

This invention was made with Government Support under Grant No. RO1DK53479, awarded by the National Institutes of Health. The Government has certain rights in the invention.

### BACKGROUND OF THE INVENTION

Obesity is an overwhelming health problem. Because of the enormous strain associated with carrying this excess weight, organs are affected, as are the nervous and circulatory systems. In 2000, the National Institute of Diabetes, Digestive and Kidney Diseases (NIDDK) estimated that there were 280,000 deaths directly related to obesity. The NIDDK further estimated that the direct cost of healthcare in the US associated with obesity is $51 billion. In addition, Americans spend $33 billion per year on weight loss products. In spite of this economic cost and consumer commitment, the prevalence of obesity continues to rise at alarming rates. From 1991 to 2000, obesity in the US grew by 61%. Not exclusively a US problem, worldwide obesity ranges are also increasing dramatically.

One of the principle costs to the healthcare system stems from the co-morbidities associated with obesity. Type-2 diabetes has climbed to 7.3% of the population. Of those persons with Type-2 diabetes, almost half are clinically obese, and two thirds are approaching obese. Other co-morbidities include hypertension, coronary artery disease, hypercholesteremia, sleep apnea and pulmonary hypertension.

The creatine kinase/creatine phosphate system is an energy generating system operative predominantly in the brain, muscle, heart, retina, adipose tissue and the kidney (Walliman et. al., Biochem. J. 281: 21-40 (1992)). The components of the system include the enzyme creatine kinase (CK), the substrates creatine (Cr), creatine phosphate (CrP), ATP, ADP, and the creatine transporter. The enzyme catalyses reversibly the transfer of a phosphoryl group from CrP to ADP to generate ATP which is the main source of energy in the cell. This system represents the most efficient way to generate energy upon rapid demand. The creatine kinase isoenzymes are found to be localized at sites where rapid rate of ATP replenishment is needed such as around ion channels and ATPase pumps. Some of the functions associated with this system include efficient regeneration of energy in the form of ATP in cells with fluctuating and high energy demand, energy transport to different parts of the cell, phosphoryl transfer activity, ion transport regulation, and involvement in signal transduction pathways.

Creatine (alpha also known as N-(aminoiminomethyl)-N-methyl glycine; methylglycosamine or N-methyl-guanidino acetic acid; see the Merck Index, Eleventh Edition No. 2570, 1989) is a compound that is naturally occurring and is found in mammalian brain, skeletal muscle, retina, adipose tissue and the heart. The phosphorylated form, creatine phosphate (see The Merck Index, No. 7315), is also found in the same organs and is the product of the creatine kinase reaction. Both compounds can be easily synthesized and are non-toxic to humans and other animals. A series of creatine analogues have also been synthesized and used as probes to study the active site of the enzyme.

Previous work on creatine analogs for the treatment of obesity and the promotion of weight loss were based on observations that systemic administration of creatine analogs induced weight loss without understanding of the mechanisms involved. However, weight reduction caused by intracellular creatine depletion occurs in parallel with side effects detrimental to the health of the subject. Systemic administration of beta-GPA, an analog of creatine and a inhibitor of intracellular creatine uptake, induces significant reductions in skeletal muscle and other muscle abnormalities (Adams et al. Journal of Applied Physiology 1994; 77: 1198-1205; Adams et al. Journal of Applied Physiology 1995; 78: 368-371; Boehm et al. American Journal of Physiology (Endocrinol. Metab) 2003; 284: E399-E406; Mahanna et al. 1980. Exp Neurol 68:114-121; Moerland et al. American Journal of Physiology (Cell Physiol. 36) 1994; 267: C127-C137; Otten et al. Metabolism 1986; 35: 481-484). Long term systemic administration of beta-GPA causes side-effects in heart function as well. (Chevli and Fitch. 1979. Biochem Med. 21:162-167; Horn et al. 2001. Circulation. 104:1844-9; Mekhi et al. 1990. Am J. Physiol. 258: H1151-H1158; Neubauer et al. 1999. J Mol Cell Cardiol. 31:1845-55; Shoubridge et al. 1985. Biochim Biophys Acta. 847: 25-32; Unitt et al. 1993. Biochim Biophys Acta. 1143: 91-96).

Accordingly, there is a need for better methods to administer creatine analogs for the promotion of weight loss without harmful systemic side effects.

### SUMMARY OF THE INVENTION

The present invention is based on the surprising discovery that inhibition of creatine uptake targeted to the hypothalamus promotes weight loss. Targeting of creatine uptake inhibition or intracellular creatine depletion to the hypothalamus circumvents the side effects of systemic creatine inhibition. Furthermore, by targeting administration of the creatine uptake inhibitor to the hypothalamus, the dose of creatine uptake inhibitor required is substantially less than the dose need when administered systemically in order to effectively promote weight loss.

In one embodiment, the invention is a method of promoting weight loss, or treating or preventing a body disorder related to excess weight, in a subject, comprising administering intracranially to the subject an effective amount of a creatine uptake inhibitor.

In one embodiment, the creatine uptake inhibitors of the invention may be indirectly administered to brain, including the hypothalamus. The creatine uptake inhibitor may comprise a brain targeting moiety, such as an anti-insulin receptor antibody, anti-transferrin receptor antibodies or activated T-cells.

In one embodiment, the invention is a method of screening for a novel compound that modulates creatine uptake in the hypothalamus, comprising administering a test compound to a test subject and measuring resulting creatine transporter expression.

### BRIEF DESCRIPTION OF THE DRAWINGS

FIG. 1A-1B shows the effect of icv administration of beta-GPA on body weight and food intake in rats. Adult male rats were implanted with indwelling stainless steel cannulae in the III ventricle of the brain and housed individually in metabolic cages as described in Methods. On Day 0 (arrow), animals were injected icv with saline (n=5; circles) or beta-GPA 2 mg (n=6; squares). Daily body weights (mean+SEM) are presented in FIG. 1A; daily food intakes (mean+SEM) are presented in FIG. 1B.

FIG. 2A-2B shows the effect of repeated icv administration of beta-GPA on body weight gain and food intake in rats. Adult male rats were implanted with indwelling stainless steel cannulae in the III ventricle of the brain and housed individually in metabolic cages as described in Methods. On days 0 and 7 (arrow), animals were injected icv with saline (n=6; circles), or beta-GPA 1 mg (n=6; squares), 2 mg (n=6; triangles) or 3 mg (n=6; x-x). Daily weights and food intakes of rats were recorded. Because the mean starting weights of the four rat groups were different, all data were normalized to a percentage of each representative groups' initial mean body weight. These normalized weights (mean+SEM) are plotted against time in FIG. 2A. Normalized food intakes (mean+SEM) are plotted against time in FIG. 2B. Analysis of the food intake data revealed significant (p=0.027) group differences with a significant (p=0.014) linear trend to the group differences (reported measures ANOVA and orthogonal polynomial trend analysis).

FIG. 3 shows the effect of icv creatine in reversing icv beta-GPA-induced weight loss in rats. Adult male rats were implanted with III ventricular cannulae as described in Methods. After surgeries, animals were handled daily for 7 days and acclimated to being confined within a small box barely larger than the rats themselves to limit movement and simulate injection conditions. On Day 0 (large arrow), all animals were injected icv with beta-GPA 2 mg while awake and confined within the small box. A second icv injection (small arrow) was administered immediately after the beta-GPA injection (within 1 min.) of either saline (n=10; circles) or creatine 0.5 mg (n=10; squares). Conscious icv injections were repeated on each of the 2 subsequent days (small arrow) with additional equal doses of saline only or creatine only, consistent with their initial injections. Body weights were recorded daily and are presented over time (mean+SEM).

FIG. 4 shows the effect of icv beta-GPA on creatine transporter mRNA in rat hypothalami. Adult male rats were injected icv (as described in Methods) with saline (n=12) or beta-GPA (n=6; |−|), on Day 0 (arrow). On day 1, food was removed from 6 of the 12 saline-treated rats to form 2 groups: saline-fed (n=6; circles) and saline-fasted (n=6; triangles). On Day 2 (arrow), animals were reinjected icv as on day 0 except that the dosage of beta-GPA was increased to 3 mg/rat. Animal weights were recorded daily and are plotted against time. Mean weights of the beta-GPA-treated and saline-fasted groups were both significantly different from that of control rats on Day 3 (*=p<0.025 utilizing Students t Test). On Day 3, rats were decapitated and hypothalamic blocks dissected and utilized to extract total RNA prior to performing Real-Time PCR as described in Methods. Fluorescent intensity of CrT amplification products were normalized to that of the rat ribosomal housekeeping gene, L32 to yield arbitrary units of gene expression. Means+SEM are presented on the graph next to each respective group. There was no statistically significant difference in CrT mRNA concentrations between treatment groups using single factor ANOVA.

FIG. 5 shows weight loss and differential display in CoPP-treated and fasted rats. Adult male Sprague Dawley rats were implanted with indwelling stainless steel cannulae and injected icv (arrow) with vehicle or CoPP 0.4 um/kg body weight (squares; n=11). Half of the vehicle-treated animals were fed ad lib (diamonds; n−11) and half were fasted for 48 hours with ad lib access to water (triangles; n=10). Animals were weighed daily. Rat weights are shown for each group from Day 0, the day of injection, to Day 2 just before decapitation. As there were a range of starting weights, data are normalized to the percentage of baseline weights in each respective group (means+SEM). Total RNA was isolated from hypothalamic blocks and subjected to reverse transcription-polymerase chain amplification as described in Methods. After separation on sequencing gels, radioactive bands were compared for differences in intensity.

FIG. 6 shows the effects of CoPP and fasting on in situ hybridization of creatine transporter mRNA in rat brain. Male rats were treated icv with vehicle or CoPP 0.4 um/kg body weight; a subset of the vehicle-treated rats was starved. After 48 hours, animals were killed and their brains extracted, sectioned and processed as described in Methods for in situ hybridization. After quantification using the Metamorph system, the areas of hybridization are expressed as percentages of the total area of the region of interest. Means+SEM of CoPP-treated (shaded bars, n=5), vehicle-treated (open bars, n=2) and vehicle-treated starved (closed bars, n=2) are plotted. Arc=arcuate nucleus, DMH=dorsomedial nucleus, VMH=ventromedial hypothalamic nucleus, 3V=3rd ventricle, Hb=habenular nucleus, PVP=paraventricular thalamic nucleus. *=p<0.05 versus control (Kruskal-Wallis Test).

FIG. 7 shows the effect of CoPP on creatine transporter immunoreactivity in rat brain. Male adult rats were treated with vehicle (closed bars), vehicle and fasted (open bars) or CoPP 0.4 umol/kg body weight (shaded bars); three animals were used in each group. Four hours after icv injections, animals were killed and immunohistochemistry carried out as described in Methods. For quantitation, areas of interest were outlined with an electronic pen and the percentage of training estimated using the Metamorph software quantitation system. Means+SEM of percentages of each area stained with fluorescent antibody are presented. Hb=habenular nucleus, PVP=paraventricular thalamic nucleus, SUM=Supramemillary nucleus, 3V=third ventricle, DMH=Dorsomedial nucleus of the hypothalamus. *=p<0.05 (Kruskal Wallis Test).

FIG. 8 shows the effect of creatine on CoPP-induced weight loss. Male rates were injected on Day 0 (open arrow) icv with vehicle or CoPP 0.4 um/kg body weight. Immediately following this injection, a second icv injection of vehicle or creatine 0.5 mg was administered which resulted in three different groups: saline-creatine (diamonds; n=6), CoPP-saline (squares; n=4) and CoPP-creatine (triangles; n=6). The creatine injections were repeated (as described in Methods) in the saline-creatine and CoPP-creatine groups another nine times (indicated by the small arrows); on each occasion, CoPP-vehicle animals got repeat dosages of vehicle. Means+SEM of animal weights are presented.

### DETAILED DESCRIPTION OF THE INVENTION

The present invention utilizes the discovery that inhibition of creatine uptake in the hypothalamus and/or reduction of intracellular creatine concentrations in the hypothalamus promotes weight loss.

### Method of Screening for a Compound Inhibiting Creatine Uptake in the Hypothalamus.

In one embodiment a method for screening a compound promoting weight loss screens for a compound that causes a reduction in intracellular creatine concentration in the hypothalamus. Greater than 90% of the intracellular creatine uptake occurs via the creatine transporter protein (GenBank accession NM_{—}005629; SLC6A8). Regulation of the creatine transporter is important in modulating creatine uptake and thus intracellular creatine concentrations (for review, see Snow and Murphy. 2001. Mol Cell Bio. 224:169-181). A marker of creatine uptake inhibition is upregulation of creatine transporter, both at the mRNA level and the protein level. Thus, in one embodiment, a method for screening a compound promoting weight loss screens for a compound that causes an increase in creatine transporter transcription or protein expression. Ablation of creatine transporter activity prevents the uptake of creatine into the cell. Thus, in another embodiment, a method for screening a compound promoting weight loss screens for a compound that inhibits creatine transporter protein in the hypothalamus.

### Test Compounds for Screening Modulators of Creatine Transporter Expression

The term “agent” or “compound” as used herein and throughout the specification means any organic or inorganic molecule, including modified and unmodified nucleic acids such as antisense nucleic acids, RNAi, such as siRNA or shRNA, peptides, peptidomimetics, receptors, ligands, and antibodies.

In the methods of the present invention, a variety of test compounds and physical conditions from various sources can be screened for the ability of the compound to alter uptake of creatine into a cell.

Compounds to be screened can be naturally occurring or synthetic molecules. Compounds to be screened can also be obtained from natural sources, such as, marine microorganisms, algae, plants, and fungi. The test compounds can also be minerals or oligo agents. Alternatively, test compounds can be obtained from combinatorial libraries of agents, including peptides or small molecules, or from existing repertories of chemical compounds synthesized in industry, e.g., by the chemical, pharmaceutical, environmental, agricultural, marine, cosmetic, drug, and biotechnological industries. Test compounds can include, e.g., pharmaceuticals, therapeutics, agricultural or industrial agents, environmental pollutants, cosmetics, drugs, organic and inorganic compounds, lipids, glucocorticoids, antibiotics, peptides, proteins, sugars, carbohydrates, chimeric molecules, and combinations thereof.

Combinatorial libraries can be produced for many types of compounds that can be synthesized in a step-by-step fashion. Such compounds include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines and oligocarbamates. In the method of the present invention, the preferred test compound is a small molecule, nucleic acid and modified nucleic acids, peptide, peptidomimetic, protein, glycoprotein, carbohydrate, lipid, or glycolipid. Preferably, the nucleic acid is DNA or RNA.

Large combinatorial libraries of compounds can be constructed by the encoded synthetic libraries (ESL) method described in Affymax, WO 95/12608, Affymax WO 93/06121, Columbia University, WO 94/08051, Pharmacopeia, WO 95/35503 and Scripps, WO 95/30642 (each of which is incorporated herein by reference in its entirety for all purposes). Peptide libraries can also be generated by phage display methods. See, e.g., Devlin, WO 91/18980. Compounds to be screened can also be obtained from governmental or private sources, including, e.g., the DIVERSet E library (16,320 compounds) from ChemBridge Corporation (San Diego, Calif.), the National Cancer Institute's (NCI) Natural Product Repository, Bethesda, Md., the NCI Open Synthetic Compound Collection, Bethesda, Md., NCI's Developmental Therapeutics Program, or the like.

Additionally, natural and synthetically produced libraries and compounds are readily modified through conventional chemical, physical, and biochemical means. In addition, known pharmacological agents may be subject to directed or random chemical modifications, such as acylation, alkylation, esterification, amidification, etc.

The compound formulations may conveniently be presented in unit dosage form, e.g., tablets and sustained release capsules, and in liposomes, and may be prepared by any methods well know in the art of pharmacy. (See, for example, Remington: The Science and Practice of Pharmacy by Alfonso R. Gennaro (Ed.) 20th edition, Dec. 15, 2000, Lippincott, Williams & Wilkins; ISBN: 0683306472.).

Screening compounds for potential effectiveness in modulating creatine transporter transcription and/or protein expression can be accomplished by a variety of means well known by a person skilled in the art.

To screen the compounds described above for ability to modulate creatine transporter transcription and/or expression, the test compounds should be administered to the test subject. In one embodiment the test subject is a culture of cells comprised of cells derived from the hypothalamus. The cells derived from the hypothalamus may be a primary cell culture or an immortalized cell line from a normal or a tumorous hypothalamus. In another embodiment, the test subject is a hypothalamus brain slice. In another embodiment, the test subject is an animal with a hypothalamus. The animal with a hypothalamus can be, but is not limited to, a frog, a rodent such as a mouse or a rat, a rabbit, a non-human primate, and a human. The hypothalamus derived cells and the hypothalamus brain slice can be obtained from the hypothalamus of a vertebrate, including but not limited to, a frog, a rodent such as a mouse or a rat, a rabbit, a non-human primate and a human.

The test compounds can be administered, for example, by diluting the compounds into the medium wherein the cell is maintained, mixing the test compounds with the food or liquid of the animal with a hypothalamus, topically administering the compound in a pharmaceutically acceptable carrier on the animal with a hypothalamus, using three-dimensional substrates soaked with the test compound such as slow release beads and the like and embedding such substrates into the animal, intracranially administering the compound, parenterally administering the compound.

A variety of other reagents may also be included in the mixture. These include reagents such as salts, buffers, neutral proteins, e.g. albumin, detergents, etc. which may be used to facilitate optimal protein-protein and/or protein-nucleic acid binding and/or reduce non-specific or background interactions, etc. Also, reagents that otherwise improve the efficiency of the assay, such as protease inhibitors, nuclease inhibitors, antimicrobial agents, etc. may be used.

The language “pharmaceutically acceptable carrier” is intended to include substances capable of being coadministered with the compound and which allows the active ingredient to perform its intended function of preventing, ameliorating, arresting, or eliminating a disease(s) of the nervous system. Examples of such carriers include solvents, dispersion media, adjuvants, delay agents and the like. The use of such media and agents for pharmaceutically active substances is well known in the art. Any conventional media and agent compatible with the compound may be used within this invention.

The compounds can be formulated according to the selected route of administration. The addition of gelatin, flavoring agents, or coating material can be used for oral applications. For solutions or emulsions in general, carriers may include aqueous or alcoholic/aqueous solutions, emulsions or suspensions, including saline and buffered media. Parenteral vehicles can include sodium chloride, potassium chloride among others. In addition intravenous vehicles can include fluid and nutrient replenishers, electrolyte replenishers among others.

Preservatives and other additives can also be present. For example, antimicrobial, antioxidant, chelating agents, and inert gases can be added (see, generally, Remington's Pharmaceutical Sciences, 16th Edition, Mack, 1980).

Screening for a compound that causes an increase in creatine transporter transcription or protein expression or screening for a compound that ablates creatine transporter activity can be accomplished using measurements of creatine transporter gene transcription and/or measurements of creatine transporter protein expression. Measurements of creatine transporter gene transcription can include direct measurements of creatine transporter gene transcription or measurements of a reporter gene. Similarly, measurements of creatine transporter protein expression can include measurements of creatine transporter protein or measurements of a reporter gene.

To test the transcription and/or protein expression of the creatine transporter or the reporter gene, a biological sample must be obtained from the test subject. A “biological sample” refers to a cell or population of cells or a quantity of tissue or fluid from an animal. Most often, the sample has been removed from an animal, but the term “biological sample” can also refer to cells or tissue analyzed in vivo, i.e. without removal from the animal. Often, a “biological sample” will contain cells from the animal, but the term can also refer to non-cellular biological material, such as non-cellular fractions of blood, saliva, or urine, that can be used to measure gene expression levels. Biological samples include, but are not limited to, tissue biopsies, scrapes (e.g. buccal scrapes), whole blood, plasma, serum, urine, saliva, cell culture, or cerebrospinal fluid. Preferred biological samples include tissue biopsies, cell culture. The sample can be obtained by removing a sample of cells from an animal, but can also be accomplished by using previously isolated cells (e.g. isolated by another person), or by performing the methods of the invention in vivo. In a preferred embodiment, the biological sample is the hypothalamus or a portion thereof.

As noted above, screening assays are generally carried out in vitro, for example, in cultured cells, in a biological sample (e.g., brain, hypothalamus derived cell culture, hypothalamus derived brain slice), or fractions thereof. For ease of description, cell cultures, biological samples, and fractions are referred to as “samples” below. The sample is generally derived from an animal (e.g., any of the research animals mentioned above), preferably a mammal, and more preferably from a human.

The reporter gene assay (Tamura, et al., Transcription Factor Research Method, Yodosha, 1993) is a method for assaying the regulation of gene expression using as the marker the expression of a reporter gene. Generally, gene expression is regulated with a part called promoter region existing in the 5′-upstream region thereof. The gene expression level at the stage of transcription can be estimated by assaying the activity of the promoter. When a test substance activates a promoter, the transcription of the reporter gene arranged downstream the promoter region is activated. In such manner, the expression of the reporter gene can be detected in place of the promoter-activating action, namely the action of activating the expression. Thus, the expression of the reporter gene can be detected in place of the action of a test substance on the regulation of the expression of the creatine transporter, by the reporter gene assay using the promoter regions of creatine transporter gene. As the “reporter gene” to be fused to the creatine transporter gene promoter region, any reporter gene for general use is satisfactory with no specific limitation. For example, an enzyme gene readily assayable quantitatively is preferable. For example, the reporter gene includes chloramphenicol acetyltransferase gene (CAT) derived from bacteria transpozon, luciferase gene (Luc) derived from firefly and green fluorescence protein gene (GFP) derived from jellyfish. The reporter gene may satisfactorily be fused functionally to creatine transporter gene promoter region. By comparing between the expression level of the reporter gene in case that a test substance is in contact with a cell transformed with the reporter gene fused to the creatine transporter gene promoter region and the expression level thereof in case that a test substance is not in contact with the reporter gene, the change of the transcription induction activity depending on the test substance can be analyzed. By carrying out the last step, screening a substance inhibiting or inducing the expression of creatine transporter can be accomplished.

Detection and quantification of the creatine transporter gene expression may be carried out through direct hybridization based assays or amplification based assays. The hybridization based techniques for measuring gene transcript are known to those skilled in the art (Sambrook et al., Molecular Cloning: A Laboratory Manual, 2d Ed. vol. 1-3, Cold Spring Harbor Press, NY, 1989). For example, one method for evaluating the presence, absence, or quantity of the creatine transporter gene is by Northern blot. Isolated mRNAs from a given biological subject are electrophoresed to separate the mRNA species, and transferred from the gel to a membrane, for example, a nitrocellulose or nylon filter. Labeled creatine transporter probes are then hybridized to the membrane to identify and quantify the respective mRNAs. The example of amplification based assays include RT-PCR, which is well known in the art (Ausubel et al., Current Protocols in Molecular Biology, eds. 1995 supplement). In a preferred embodiment, quantitative RT-PCR is used to allow the numerical comparison of the level of respective creatine transporter mRNAs in different samples. A Real-Time or TaqMan-based assay also can be used to quantify creatine transporter gene transcription.

All of the following gene transcription and polypeptide or protein expression assays can be used to detect either the creatine transporter transcription and/or expression. Alternatively, when a reporter gene is utilized, the transcription and/or expression of the reporter gene may also be detected in place of the creatine transporter gene utilizing the following amplification based, hybridization based and polypeptide based assays.

### Amplification-Based Assays

In one embodiment, amplification-based assays can be used to detect, and optionally quantify, a polynucleotide encoding creatine transporter. In such amplification-based assays, the creatine transporter mRNA in the sample act as template(s) in an amplification reaction carried out with a nucleic acid primer that contains a detectable label or component of a labeling system. Suitable amplification methods include, but are not limited to, polymerase chain reaction (PCR); reverse-transcription PCR (RT-PCR); ligase chain reaction (LCR) (see Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

To determine the level of the creatine transporter mRNA, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990).

Real-time PCR is also useful to study the changing levels of expression of genetic sequences over time. Real-time PCR using the TaqMan (registered trade mark) system developed by PE Biosystems (Foster City, Calif., USA) allows rapid detection and quantitation of DNA without the need for labour intensive post-PCR processing such as gel electrophoresis and radioactive hybridization (Heid et al., 1996). In addition, the built-in 96-well format greatly increases the number of samples which can be simultaneously analyzed. The method uses the 5′ exonuclease activity of a Taq polymerase (AmpliTaq Gold, PE Biosystems, Foster City, Calif., USA) during primer extension to cleave a dual-labelled, fluorogenic probe hybridized to the target DNA between the PCR primers. Prior to cleavage, a reporter dye, such as 6-carboxyfluorescein (6-FAM) at the 5′ end of the probe is quenched by 6-carboxy-tetramethylrhodamine (TAMRA) through fluorescent resonance energy transfer. Following digestion, FAM is released. The resulting fluorescence is continuously measured in real-time at 518 nm during the log phase of product accumulation and is proportional to the number of copies of the target sequence.

In a preferred embodiment, suitable for use in amplification-based assays of the invention, a primer contains two fluorescent dyes, a “reporter dye” and a “quencher dye.” When intact, the primer produces very low levels of fluorescence because of the quencher dye effect. When the primer is cleaved or degraded (e.g., by exonuclease activity of a polymerase, see below), the reporter dye fluoresces and is detected by a suitable fluorescent detection system. Amplification by a number of techniques (PCR, RT-PCR, RCA, or other amplification method) is performed using a suitable DNA polymerase with both polymerase and exonuclease activity (e.g., Taq DNA polymerase). This polymerase synthesizes new DNA strands and, in the process, degrades the labeled primer, resulting in an increase in fluorescence. Commercially available fluorescent detection systems of this type include the ABI Prism Systems 7000, 7700, or 7900 (TaqMan) from Applied Biosystems or the LightCycler System from Roche.

### Hybridization-Based Assays

Nucleic acid hybridization simply involves contacting a nucleic acid probe with sample polynucleotides under conditions where the probe and its complementary target nucleotide sequence can form stable hybrid duplexes through complementary base pairing. The nucleic acids that do not form hybrid duplexes are then washed away leaving the hybridized nucleic acids to be detected, typically through detection of an attached detectable label or component of a labeling system. Methods of detecting and/or quantifying polynucleotides using nucleic acid hybridization techniques are known to those of skill in the art (see Sambrook et al. supra). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587. Methods of optimizing hybridization conditions are described, e.g., in Tijssen (1993) Laboratory Techniques in Biochemistry and Molecular Biology, Vol. 24: Hybridization With Nucleic Acid Probes, Elsevier, N.Y.).

The nucleic acid probes used herein for detection of creatine transporter mRNA can be full-length or less than the full-length of these polynucleotides. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of the channel subunit mRNA of interest, and standard methods (such as, e.g., densitometry where the nucleic acid probe is radioactively labeled) can be used to quantify the level of the creatine transporter polynucleotide.)

A variety of additional nucleic acid hybridization formats are known to those skilled in the art. Standard formats include sandwich assays and competition or displacement assays. Sandwich assays are commercially useful hybridization assays for detecting or isolating polynucleotides. Such assays utilize a “capture” nucleic acid covalently immobilized to a solid support and a labeled “signal” nucleic acid in solution. The sample provides the target polynucleotide. The capture nucleic acid and signal nucleic acid each hybridize with the target polynucleotide to form a “sandwich” hybridization complex.

In one embodiment, the methods of the invention can be utilized in array-based hybridization formats. In an array format, a large number of different hybridization reactions can be run essentially “in parallel.” This provides rapid, essentially simultaneous, evaluation of a number of hybridizations in a single experiment. Methods of performing hybridization reactions in array based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low-density” arrays can simply be produced by spotting (e.g. by hand using a pipette) different nucleic acids at different locations on a solid support (e.g. a glass surface, a membrane, etc.). This simple spotting approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

Hybridization assays according to the invention can also be carried out using a MicroElectroMechanical System (MEMS), such as the Protiveris' multicantilever array.

Creatine transporter RNA is detected in the above-described polynucleotide-based assays by means of a detectable label. Any of the labels discussed above can be used in the polynucleotide-based assays of the invention. The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

### Polypeptide-Based Assays

Creatine transporter polypeptides can be detected and quantified by any of a number of methods well known to those of skill in the art. Examples of analytic biochemical methods suitable for detecting creatine transporter protein include electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunohistochemistry, affinity chromatography, immunoelectrophoresis, radioimmunoassay (RIA), enzyme-linked immunosorbent assays (ELISAs), immunofluorescent assays, Western blotting, and the like.

Creatine transporter polypeptide (or fragments thereof) can be detected and quantified using various well-known immunological assays. Immunological assays refer to assays that utilize an antibody (e.g., polyclonal, monoclonal, chimeric, humanized, scFv, and fragments thereof) that specifically binds to creatine transporter polypeptide (or a fragment thereof). A number of well-established immunological assays suitable for the practice of the present invention are known, and include ELISA, radioimmunoassay (RIA), immunoprecipitation, immunofluorescence, and Western blotting.

The anti-creatine transporter antibodies (preferably anti-mammalian creatine transporter; more preferably anti-human creatine transporter) to be used in the immunological assays of the present invention are commercially available from, e.g., Alpha Diagnostics (San Antonio, Tex.) and Research Diagnostics (Flanders, N.J.). Alternatively, creatine transporter antibodies may be produced by methods well known to those skilled in the art. For example, monoclonal antibodies to creatine transporter (preferably mammalian; more preferably human can be produced by generation of hybridomas in accordance with known methods. Hybridomas formed in this manner are then screened using standard methods, such as ELISA, to identify one or more hybridomas that produce an antibody that specifically binds to creatine transporter. Full-length creatine transporter may be used as the immunogen, or, alternatively, antigenic peptide fragments of creatine transporter may be used.

As an alternative to preparing monoclonal antibody-secreting hybridomas, a monoclonal antibody to creatine transporter may be identified and isolated by screening a recombinant combinatorial immunoglobulin library (e.g., an antibody phage display library) to thereby isolate immunoglobulin library members that bind to creatine transporter. Kits for generating and screening phage display libraries are commercially available from, e.g., Dyax Corp. (Cambridge, Mass.) and Maxim Biotech (South San Francisco, Calif.). Additionally, examples of methods and reagents particularly amenable for use in generating and screening antibody display libraries can be found in the literature.

Polyclonal sera and antibodies may be produced by immunizing a suitable subject, such as a rabbit, with creatine transporter (preferably mammalian; more preferably human) or an antigenic fragment thereof. The antibody titer in the immunized subject may be monitored over time by standard techniques, such as with ELISA, using immobilized marker protein. If desired, the antibody molecules directed against creatine transporter may be isolated from the subject or culture media and further purified by well-known techniques, such as protein A chromatography, to obtain an IgG fraction.

Fragments of antibodies to creatine transporter may be produced by cleavage of the antibodies in accordance with methods well known in the art. For example, immunologically active F(ab′) and F(ab′)₂ fragments may be generated by treating the antibodies with an enzyme such as pepsin. Additionally, chimeric, humanized, and single-chain antibodies to creatine transporter, comprising both human and nonhuman portions, may be produced using standard recombinant DNA techniques. Humanized antibodies to creatine transporter may also be produced using transgenic mice that are incapable of expressing endogenous immunoglobulin heavy and light chain genes, but which can express human heavy and light chain genes.

In the immunological assays of the present invention, the creatine transporter polypeptide is typically detected directly (i.e., the anti-creatine transporter antibody is labeled) or indirectly (i.e., a secondary antibody that recognizes the anti-creatine transporter antibody is labeled) using a detectable label. The particular label or detectable group used in the assay is usually not critical, as long as it does not significantly interfere with the specific binding of the antibodies used in the assay.

The immunological assays of the present invention may be competitive or noncompetitive. In competitive assays, the amount of creatine transporter in a sample is measured indirectly by measuring the amount of added (exogenous) creatine transporter displaced from a capture agent (i.e., an anti-creatine transporter antibody) by the creatine transporter in the sample. In noncompetitive assays, the amount of creatine transporter in a sample is directly measured. In a preferred noncompetitive “sandwich” assay, the capture agent (e.g., a first anti-creatine transporter antibody) is bound directly to a solid support (e.g., membrane, microtiter plate, test tube, dipstick, glass or plastic bead) where it is immobilized. The immobilized agent then captures any creatine transporter polypeptide present in the sample. The immobilized creatine transporter can then be detected using a second labeled anti-creatine transporter antibody. Alternatively, the second anti-creatine transporter antibody can be detected using a labeled secondary antibody that recognizes the second anti-creatine transporter antibody.

### Method to Promote Weight Loss by Administration of a Creatine Uptake Inhibitor to the Hypothalamus

The present invention targets the administration of creatine uptake inhibitors to site of action for creatine uptake inhibition in the promotion of weight loss thereby circumventing the side effects of systemic creatine uptake inhibition.

The test compound obtained from the screen or known creatine uptake inhibitor described herein, can be administered therapeutically in a subject to promote weight loss. Preferred subjects include humans, non-human primates, and other mammals. Thus, it will be recognized that the methods of this invention contemplate veterinary applications as well as medical applications directed to humans. Therapeutic administration should modulate creatine uptake preferentially in the hypothalamus. In a preferred embodiment, the compound is administered intracranially to the hypothalamus.

Compounds which are particularly effective for this purpose include creatine analogs and creatine phosphate analogs which are described in detail below. The term “creatine compounds” will be used herein to include creatine, creatine phosphate, and compounds which are structurally similar to creatine or creatine phosphate, and analogs of creatine and creatine phosphate. The term “creatine compounds” also includes compounds which “mimic” the activity of creatine, creatine phosphate or creatine analogs, i.e., compounds which inhibit or modulate the creatine uptake system. The term “mimics” is intended to include compounds which may not be structurally similar to creatine but mimic the therapeutic activity of creatine, creatine phosphate or structurally similar compounds. The term “inhibitors of creatine uptake system” are compounds which inhibit the activity of the creatine kinase enzyme, molecules that inhibit the creatine transporter or molecules that inhibit the binding of the enzyme to other structural proteins or enzymes or lipids. The term “modulators of the creatine uptake system” are compounds which modulate the activity of the enzyme, or the activity of the transporter of creatine or the ability of other proteins or enzymes or lipids to interact with the system. The term “creatine analog” is intended to include compounds which are structurally similar to creatine or creatine phosphate, compounds which are art-recognized as being analogs of creatine or creatine phosphate, and/or compounds which share the same or similar function as creatine or creatine phosphate.

Most of these compounds have been previously synthesized for other purposes (Rowley et. al., J. Am. Chem. Soc., 93: 5542-5551, (1971); Mclaughlin et. al., J. Biol. Chem., 247: 4382-4388 (1972) Nguyen, A. C. K., “Synthesis and enzyme studies using creatine analogues”, Thesis, Dept of Pharmaceutical Chemistry, Univ. Calif., San Francisco, 1983; Lowe et al., J. Biol. Chem., 225:3944-3951 (1980); Roberts et. al., J. Biol. Chem., 260:13502-13508 (1995) Roberts et. al., Arch. biochem. Biophy., 220:563-571, 1983, and Griffiths et. al., J. Biol. Chem., 251: 2049-2054 (1976). The contents of all of the forementioned references are expressly incorporated by reference. Further to the forementioned references, Kaddurah-Daouk et. al., (WO 92/08456; WO 90/09192; U.S. Pat. No. 5,324,731; U.S. Pat. No. 5,321,030; U.S. Pat. App. No. 2004/0116390) also provide citations for the synthesis of a plurality of creatine analogs. The contents of all the aforementioned references and patents are incorporated herein by reference.

Examples of useful creatine analogs, e.g., inhibitors of creatine uptake, include, for example, cyclocreatine, homocyclocreatine, beta guanidino propionic acid (β-GPA), guanidinoacetate, 1-carboxymethyl-2-iminohexahydropyrimidine, guanidino acetate, carbocreatine, phosphocyclocreatine. As used herein, “creatine analogs” and “creatine uptake inhibitors” are used interchangeably.

Creatine analogs which are particularly useful in this invention include those encompassed by the following general formula:

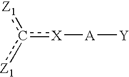

and pharmaceutically acceptable salts thereof, wherein:
a) Y is selected from the group consisting of: —CO₂H—NHOH, —NO₂, —SO₃H, —C(═O)NHSO₂J and —P(═O)(OH)(OJ), wherein J is selected from the group consisting of: hydrogen, C₁-C₆ straight chain alkyl, C₃-C₆ branched alkyl, C₂-C₆ alkenyl, C₃-C₆ branched alkenyl, and aryl;
b) A is selected from the group consisting of C, CH, C₁-C₅alkyl, C₂-C₅alkenyl, C₂-C₅alkynyl, and C₁-C₅alkoyl chain, each having 0-2 substituents which are selected independently from the group consisting of
   1) K, where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: rromo, chloro, epoxy and acetoxy;
   2) an aryl group selected from the group consisting of a 1-2 ring carbocycle and a 1-2 ring heterocycle, wherein the aryl group contains 0-2 substituents independently selected from the group consisting of —CH₂L and —COCH₂L where L is independently selected from the group consisting of bromo, chloro, epoxy and acetoxy; and
   3) —NH-M, wherein M is selected from the group consisting of: hydrogen, C₁-C₄ alkyl, C₂-C₄ alkenyl, C₁-C₄ alkoyl, C₃-C₄ branched alkyl, C₃-C₄ branched alkenyl, and C₄ branched alkoyl;
c) X is selected from the group consisting of NR₁, CHR₁, CR₁, O and S, wherein R₁ is selected from the group consisting of:

1) hydrogen;

2) K where K is selected from the group consisting of: C₁-C₆ straight alkyl, C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;
3) an aryl group selected from the group consisting of a 1-2 ring carbocycle and a 1-2 ring heterocycle, wherein the aryl group contains 0-2 substituents independently selected from the group consisting of —CH₂L and —COCH₂L where L is independently selected from the group consisting of: bromo, chloro, epoxy and acetoxy;
4) a C₅-C₈ alpha-amino-omega-methyl-omega-adenosylcarboxylic acid attached via the w-methyl carbon,
5) 2 C₅-C₉ alpha-amino-omega-aza-omega-methyl-w-adenosylcarboxylic acid attached via the omega-methyl carbon; and
6) a C₅-C₉ alpha-amino-omega-thia-omega-methyl-omega-adenosylcarboxylic acid attached via the w-methyl carbon;
d) Z₁ and Z₂ are chosen independently from the group consisting of: ═O, —NHR₂, —CH₂R₂, —NR₂OH; wherein Z₁ and Z₂ may not both be ═O and wherein R₂ is selected from the group consisting of:
   1) hydrogen;
   2) K, where K is selected from the group consisting of: C₁-C₆ straight alkyl; C₂-C₆ straight alkenyl, C₁-C₆ straight alkoyl, C₃-C₆ branched alkyl, C₃-C₆ branched alkenyl, and C₄-C₆ branched alkoyl, K having 0-2 substituents independently selected from the group consisting of: bromo, chloro, epoxy and acetoxy;
   3) an aryl group selected from the group consisting of a 1-2 ring carbocycle and a 1-2 ring heterocycle, wherein the aryl group contains 0-2 substituents independently selected from the group consisting of: —CH₂L and —COCH₂L where L is independently selected from the group consisting of: bromo, chloro, epoxy and acetoxy;
   4) 2 C₄-C₈ alpha-amino-carboxylic acid attached via the w-carbon;
   5) B, wherein B is selected from the group consisting of: —CO₂H—NHOH, —SO₃H, —NO₂, OP(═O)(OH)(OJ) and —P(═O)(OH)(OJ), wherein J is selected from the group consisting of: hydrogen, C₁-C₆ straight allyl, C₃-C₆ branched alkyl, C₂-C₆ alkenyl, C₃-C₆ branched alkenyl, and aryl, wherein B is optionally connected to the nitrogen via a linker selected from the group consisting of: C₁-C₂ alkyl, C₂ alkenyl, and C₁-C₂ alkoyl;
   6)-D-E, wherein D is selected from the group consisting of: C₁-C₃ straight alkyl, C₃ branched alkyl, C₂-C₃ straight alkenyl, C₃ branched alkenyl, C₁-C₃ straight alkoyl, aryl and aroyl; and E is selected from the group consisting of —(PO₃) ₙNMP, where n is 0-2 and NMP is ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O)(OH)(CH₂)]ₘ-Q, where m is 0-3 and Q is a ribonucleoside connected via the ribose or the aromatic ring of the base; and an aryl group containing 0-3 substituents chosen independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl, wherein E may be attached to any point to D, and if D is alkyl or alkenyl, D may be connected at either or both ends by an amide linkage; and
   7)-E, wherein E is selected from the group consisting of —(PO₃)ₙNMP, where n is 0-2 and NMP is a ribonucleotide monophosphate connected via the 5′-phosphate, 3′-phosphate or the aromatic ring of the base; —[P(═O)(OCH₃)(O)]ₘ-Q, where m is 0-3 and Q is a ribonucleoside connected via the ribose or the aromatic ring of the base; —[P(═O(O)(OH)(CH₂)]ₘ-Q, where m is 0-3 and Q is a ribonucleoside connected via the ribose or the aromatic ring of the base; and an aryl group containing 0-3 substituents chose independently from the group consisting of: Cl, Br, epoxy, acetoxy, —OG, —C(═O)G, and —CO₂G, where G is independently selected from the group consisting of: C₁-C₆ straight alkyl, C₂-C₆ straight alkenyl, C₁-C₆ straight alkoyl, C₃-C₆ branched alkyl, C₃-C₆ branched alkenyl, C₄-C₆ branched alkoyl; and if E is aryl, E may be connected by an amide linkage;
e) if R₁ and at least one R₂ group are present, R₁ may be connected by a single or double bond to an R₂ group to form a cycle of 5 to 7 members;
f) if two R₂ groups are present, they may be connected by a single or a double bond to form a cycle of 4 to 7 members; and
g) if R₁ is present and Z₁ or Z₂ is selected from the group consisting of —NHR₂, —CH₂R₂ and —NR₂OH, then R₁ may be connected by a single or double bond to the carbon or nitrogen of either Z₁ or Z₂ to form a cycle of 4 to 7 members.

The creatine analogs described above may be distinguished between those that promote weight loss and those that promote weight gain by testing the effects of the administration thereof in culture or in an animal model, as described in the examples, or any other method known to the skilled artisan.

Methods for delivery of an agent to a discrete area of the brain are well known in the art, and can include the use of stereotactic imaging and delivery devices.

The present invention encompasses any suitable method for intracranial administration of a creatine uptake inhibitor to a selected target tissue, including injection of an aqueous solution of a creatine uptake inhibitor and implantation of a controlled release system, such as a creatine uptake inhibitor incorporating polymeric implant at the selected target site. Use of a controlled release implant reduces the need for repeat injections. Intracranial implants are known. For example, brachytherapy for malignant gliomas can include stereotatically implanted, temporary, iodine-125 interstitial catheters. Scharfen. C. O., et al., High Activity Iodine-125 Intersitial Implant For Gliomas, Int. J. Radiation Oncology Biol Phys 24(4); 583-591:1992. Additionally, permanent, intracranial, low dose I-125 seeded catheter implants have been used to treat brain tumors. Gaspar, et al., Permanent I-125 Implants for Recurrent Malignant Gliomas, Int J Radiation Oncology Biol Phys 43(5); 977-982:1999. See also chapter 66, pages 577-580, Bellezza D., et al., Stereotactic Interstitial Brachytherapy, in Gildenberg P. L. et al., Textbook of Stereotactic and Functional Neurosurgery, McGraw-Hill (1998).

Furthermore, local administration of an anti cancer drug to treat malignant gliomas by interstitial chemotherapy using surgically implanted, biodegradable implants is known. For example, intracranial administration of 3-bis(chloro-ethyl)-1-nitrosourea (BCNU) (Carmustine) containing polyanhydride waters, has found therapeutic application. Brem, H. et al. The Safety of Interstitial Chemotherapy with BCNU-Loaded Polymer Followed by Radiation Therapy in the Treatment of Newly Diagnosed Malignant Gliomas: Phase I Trial, J Neuro-Oncology 26:111-123:1995.

A polyanhydride polymer, Gliadel® (Stolle R & D, Inc., Cincinnati, Ohio) a copolymer of poly-carboxyphenoxypropane and sebacic acid in a ratio of 20:80 has been used to make implants, intracranially implanted to treat malignant gliomas. Polymer and BCNU can be co-dissolved in methylene chloride and spray-dried into microspheres. The microspheres can then be pressed into discs 1.4 cm in diameter and 1.0 mm thick by compression molding, packaged in aluminum foil pouches under nitrogen atmosphere and sterilized by 2.2 megarads of gamma irradiation. The polymer permits release of carmustine over a 2-3 week period, although it can take more than a year for the polymer to be largely degraded. Brem, H., et al, Placebo-Controlled Trial of Safety and Efficacy of Intraoperative Controlled Delivery by Biodegradable Polymers of Chemotherapy for Recurrent Gilomas, Lancet 345; 10081012:1995.

Stereotactic procedures can be used for precise intracranial administration of creatine uptake inhibitor in aqueous form or as an implant. A cranial neuroblastoma is also treated in this manner. Thus, intracranial administration of a creatine uptake inhibitor can be carried out as follows.

A preliminary MRI scan of the patient can be carried out to obtain the length of the anterior commissure-posterior commissure line and its orientation to external bony landmarks. The base of the frame can then be aligned to the plane of the anterior commissure-posterior commissure line. CT guidance is used and can be supplemented with ventriculography. The posterior commissure can be visualized on 2-mm CT slices and used as a reference point.

Physiological corroboration of target tissue localization can be by use of high and low frequency stimulation through an electrode accompanying or incorporated into the long needle syringe used. A thermistor electrode 1.6 mm in diameter with a 2 mm exposed tip can be used (Radionics, Burlington, Mass.). With electrode high frequency stimulation (75 Hz) paraesthetic responses can be elicited in the forearm and hand at 0.5-1.0 V using a Radionics lesion generator (Radionics Radiofrequency Lesion Generator Model RFG3AV). At low frequency (5 Hz) activation or disruption of tremor in the affected limb occurred at 2-3 V. With the methods of the present invention, the electrode is not used to create a lesion. Following confirmation of target tissue localization, a creatine uptake inhibitor can be injected. A typical injection is the desired number of units (i.e. about 0.1 to about 5 units of a creatine uptake inhibitor in about 0.1 ml to about 0.5 ml of water or saline. A low injection volume can be used to minimize toxin diffusion away from target. Typically, the creatine uptake inhibition effect can be expected to wear off within a few days to about 2-4 months depending on the compound. For example, cobalt protoporphyrin has an exceptionally long duration of action and would be expected to last for months. In another example, beta-GPA has a duration of action of a few days. Thus, an alternate creatine uptake inhibitor format, creatine uptake inhibitor incorporated within a polymeric implant, can be used to provide controlled, continuous release of a therapeutic amount of the creatine uptake inhibitor at the desired location over a prolonged period (i.e. from about 1 year to about 6 years), thereby obviating the need for repeated creatine uptake inhibitor injections.

Several methods can be used for stereotactically guided injection of a creatine uptake inhibitor to various intracranial targets, such as the arcuate nucleus (AN) for treatment of acromegaly. Thus a stereotactic magnetic resonance (MRI) method relying on three-dimensional (3D) T1-weighted images for surgical planning and multiplanar T2-weighted images for direct visualization of the AN, coupled with electrophysiological recording and injection guidance for AN injection can be used. See e.g. Bejjani, B. P., et al., Bilateral Subthalamic Stimulation for Parkinson's Disease by Using Three-Dimensional Stereotactic Magnetic Resonance Imaging and Electrophysiological Guidance, J Neurosurg 92(4); 615-25:2000. The coordinates of the center of the AN can be determined with reference to the patient's anterior commissure-posterior commissure line and a brain atlas.

Electrophysiological monitoring through several parallel tracks can be performed simultaneously to define the functional target accurately. The central track, which is directed at the predetermined target by using MRI imaging, can be selected for neurotoxin injection. No surgical complications are expected.

Computer-aided atlas-based functional neurosurgery methodology can be used to accurately and precisely inject the desired neurotoxin or implant a neurotoxin controlled release implant. Such methodologies permit three-dimensional display and real-time manipulation of hypothalamic structures. Neurosurgical planning with mutually preregistered multiple brain atlases in all three orthogonal orientations is therefore possible and permits increased accuracy of target definition for creatine uptake inhibitor injection or implantation, reduced time of the surgical procedure by decreasing the number of tracts, and facilitates planning of more sophisticated trajectories. See for example Nowinski W. L. et al., Computer-Aided Stereotactic Functional Neurosurgery Enhanced by the Use of the Multiple Brain Atlas Database, IEEE Trans Med Imaging 19(1); 62-69:2000.

Thus, the hypothalamus or the third ventricule can be treated by local administration of from 1 to 500 units of a creatine uptake inhibitor to the target tissue.

In one embodiment, the creatine uptake inhibitors of the invention may be indirectly administered to brain, including the hypothalamus. The creatine uptake inhibitor may comprise a brain targeting moiety, such as an anti-insulin receptor antibody (Coloma et al., (2000) Pharm Res 17:266-74), anti-transferrin receptor antibodies (Zhang and Pardridge, (2001) Brain res 889:49-56) or activated T-cells (Westland et al., (1999) Brain 122:1283-91).

It will be apparent to those skilled in the art that various modifications and variations can be made to the present invention without departing from the spirit and scope of the invention. Thus, it is intended that the present invention cover the modifications and variations of this invention provided they come within the scope of the appended claims and their equivalents.

The contents of all references, patents and published patent applications cited throughout this application, as well as the Appendix, and Figures, are incorporated herein by reference in their entirety. This invention is further illustrated by the following examples which should not be construed as limiting.

### EXAMPLES

### Example 1

### Inhibition of Creatine Uptake in the Brain Leads to Hypophagia and Weight Loss in Rats

### Materials and Methods

Creatine (Cr; purity >99%) and 3-guanidopropionic acid (beta-GPA; purity >99%) were purchased from Sigma-Aldrich (St. Louis, Mo.). All other chemicals were of the highest reagent grade commercially available.

### Animal Handling and Treatment

All animal handling was approved by and done in accordance with the guidelines and requirements of the University of Vermont Institutional Animal Care Use Committee. Adult male Sprague-Dawley rats (mycoplasma free; 200 g) were purchased from Charles River (Quebec, Canada) and housed in an air-conditioned (23+1 degree C.) room with lights on for 12 hours daily (starting at 6:00 a.m.) in the University of Vermont Animal Facility. Rats were housed singly in regular cages with free access to Purina Rat Chow (RMH 3000) and water for at least five days prior to surgery. Stereotactic surgery was used to implant chronic indwelling stainless steel cannulae into the third ventricle of the brain under fentanyl/droperidol anesthesia. Coordinates for cannula placement were obtained from the Konig Klippel stereotactic surgery atlas [18]; with the nose bar 2.4 mm below the intraaural line and the vertical micrometer gantry tilted 9 degrees towards the animal, the coordinates were 4.9 mm dorsal to the intraaural line, 5 mm anterior to the intraaural line and 1.1 mm lateral to the midline [19]. A minimum of 5 days later, animals were injected icv with beta-GPA (which was dissolved in saline at 50 degrees C. and the pH adjusted to 7.2-7.4 with dilute HCl) or other experimental compounds at the indicated concentrations. Control animals received equal volumes of vehicle. Volumes of icv injections never exceeded 10 ul per animal and were administered over 5-10 seconds. In some experiments, animals received two or more icv injections on the same or different days. Measures of body weight were made daily with a digital balance with a 6 second integration period to minimize the effects of animal movement. For experiments involving measurement of food intake, after stereotactic surgery animals were housed in individual Nalgene metabolic cages and given additional time for acclimation to the cage and feeding system. Food intakes were measured using powdered rat chow with daily weigh-backs as indicated.

### Immunohistochemistry Experiments

In experiments involving immunohistochemistry, animals were injected icv with beta-GPA or with vehicle. Four hours after injection, animals were anesthetized with halothane and perfused through the left ventricle of the heart, initially with oxygenated Krebs buffer and then with ˜350 ml of 4% paraformaldehyde in 0.1M NaCl, pH 7.4 phosphate buffered saline (PBS). Brains were extracted from the skull, hypothalamic blocks dissected (by coronal cuts anterior and posterior to hypothalamic landmarks) and post-fixed in 4% paraformaldehyde solution at 4 degrees C. overnight. The following day, the blocks were transferred to 30% sucrose and incubated overnight at 4 degrees C. After freezing in Optimum Cutting Temperature (OCT) compound (Sakura, Japan), blocks were sectioned coronally at 40 um on a cryostat and sections individually floated in multi-well dishes filled with PBS. Sections were treated for 30 minutes with 0.4% Triton detergent in potassium PBS to aid in antibody penetration. Primary antibodies were added, the plates shaken gently for 1 hour at room temperature, then incubated at 4 degrees C. for 72 hours. Sections were then brought to room temperature while shaking and the primary antibody solution was removed by washing 3 times with PBS buffer. After removal of the last wash, the secondary antibody solution was added and the plates incubated for 2 hours at room temperature in the dark. Removal of the second antibody was achieved by washing three times with PBS buffer prior to mounting on subbed slides. They were allowed to dry, rewetted with PBS buffer and cover-slipped with Citifluor (Ted Pella, Reading, Calif.).

Fos studies were performed using indirect fluorescence visualization. The method utilized rabbit c-Fos-AB5 (1:2,000; Oncogene Research Products, Cambridge, Mass.) with goat anti-rabbit Cy3 (1:500; Jackson Immuno Research Laboratories, West Grove, Pa.) as the secondary antibody. Tissue incubated in the absence of primary or secondary antisera showed no reaction products. Staining observed in experimental tissue was compared to that observed from experiment-matched negative controls. Nuclei exhibiting immunoreactivity that was greater than the background level observed in experiment-matched negative controls were considered positively stained. Immunoreactive Fos (Fos IR) was quantified by capturing images at 10× using MagnaFire 2.0 Software. These images were imported into the Metamorph 6.1 Software package and density of staining measured using Hue Saturation Intensity. Regions outlining nuclei were hand drawn with an electric pen and the total areas and stained areas measured to yield percentages of total areas with specific Fos-IR. The values for left and right dorsomedial nuclei in each rat were averaged, and the averages used to calculate means. In the case of the III ventricle, the entire image was used for quantification prior to calculating the percentage of the area with Fos-IR.

### Real-Time PCR

RNA was purified with RNA-free DNase (Promega) at 37 degrees C. for 30 min. The first strand of cDNA was synthesized by adding 1 ug of purified RNA from each sample to 20 ul containing MMLV reverse transcriptase (Invitrogen Life Tech). Creatine transporter (CrT) gene expression was further quantatified by Real-Time PCR using sense primer: 5′GGTAAGGGTGCAA GCCTTTG′ (SEQ ID NO: 1); antisense primer: 5′TTGCTATGTTTGCAGTGGCT-3′ (SEQ ID NO:2) and TaqMan probe: 5′ACATTCTTACTGTGCTAAAAAAGCCACTGC-3′ (SEQ ID NO: 3) (MMG-Biotech). The rat ribosomal protein L32 gene was used as a loading control. 1 ul of each cDNA sample derived from the 20 ul reverse transcription reaction and 1 ug RNA template was mixed with 24 ul master mixture (Perkin Elmer) in a 96-well TaqMan plate. The PCR was run in an ABI prism 7700 thermocycler (Applied Biosystems) at 50 degrees C. for 2 min and 95 degrees C. for 10 min, followed by 95 degrees C. for 15 sec and 60 degrees C. for 1 min for 40 cycles. The data were analyzed using software supplied with the ABI prism 7700.

### Statistics

Experiments involving animal weights and quantification of Fos immunohistochemistry were analyzed using Students t test. Results of Real-Time PCR were analyzed utilizing single factor ANOVA.

### Results

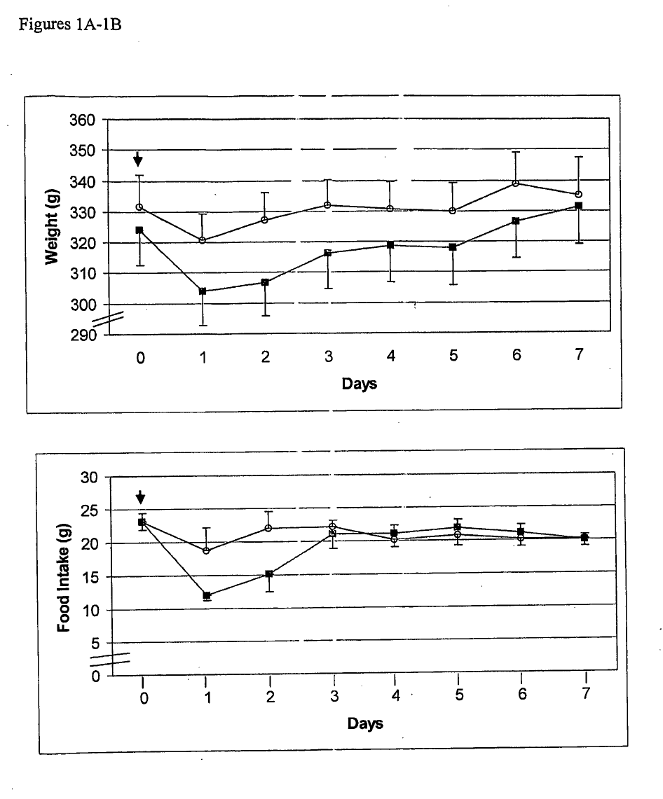

Initial dose-ranging studies were conducted using icv administration of beta-GPA. Oral administration by adding 1% beta-GPA to the chow delivers a daily systemic dose of approximately 200 mg. The targeted icv dosage, about 100-fold less, was therefore 2 mg per rat. A five-fold greater dose (10 mg) was not usable because of solubility dictates and a five-fold lower dose (0.04 mg) was without effect. However, the calculated dose of 2 mg/rat, as shown in FIG. 1, elicited modest decreases in both body weight and food intake; both indices returned to values indistinguishable from those observed in vehicle-treated animals within four days of treatments.

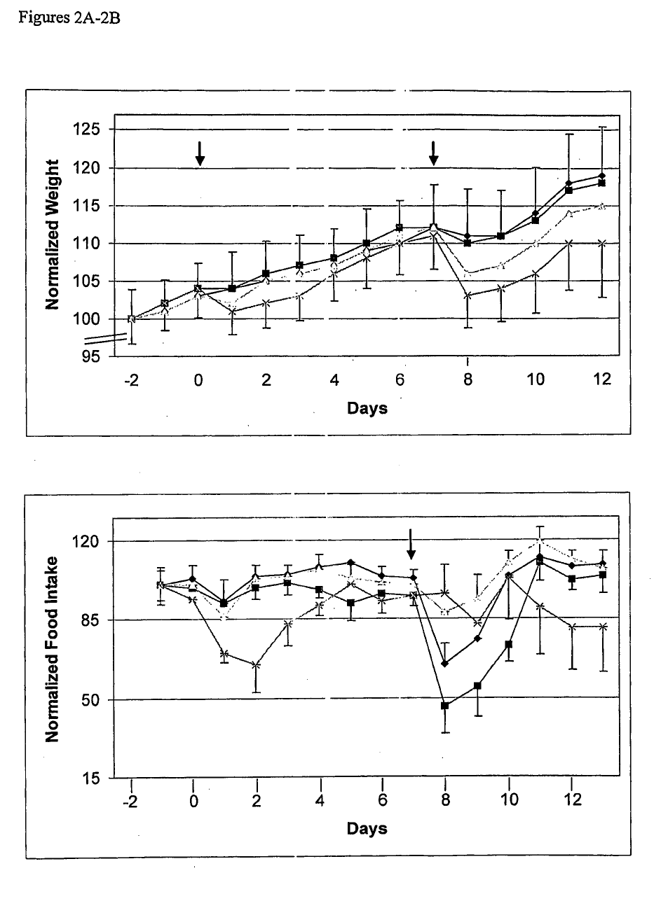

Body weight and food intake decreased in a dose-dependent fashion following icv administration of beta-GPA 1 mg, 2 mg, or 3 mg per animal (FIG. 2). However, the magnitude of both effects, but particularly that of the weight changes, was enhanced considerably following a second identical dosage of beta-GPA seven days after the first treatment (FIG. 2).

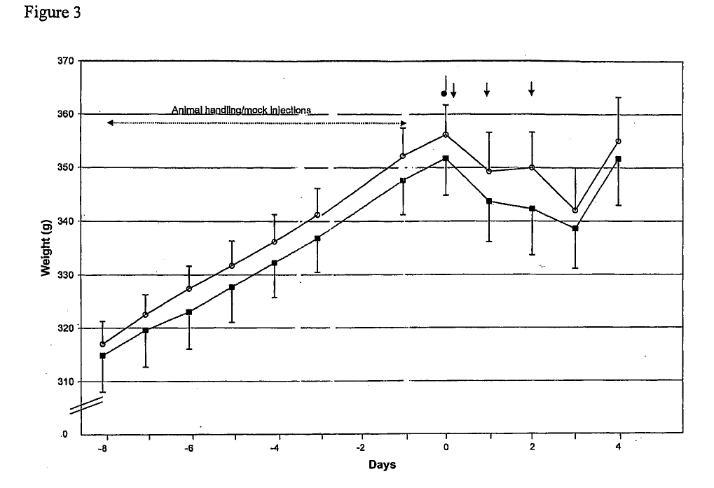

In view of the reported effects of beta-GPA in reducing the cellular contents of Cr and PCr [3], experiments were conducted to determine if icv administration of creatine could modify or reverse the weight loss induced by icv treatment with beta-GPA. As shown in FIG. 3, the weight loss induced by icv administration of beta-GPA (2 mg/rat) was not modified by co-administration of creatine 0.5 mg/rat. Subsequent repeat icv injections of creatine 0.5 mg/rat on each of the following two days was also without effect on weight loss which was indistinguishable from that observed in beta-GPA-treated rats administered vehicle injections instead of creatine.

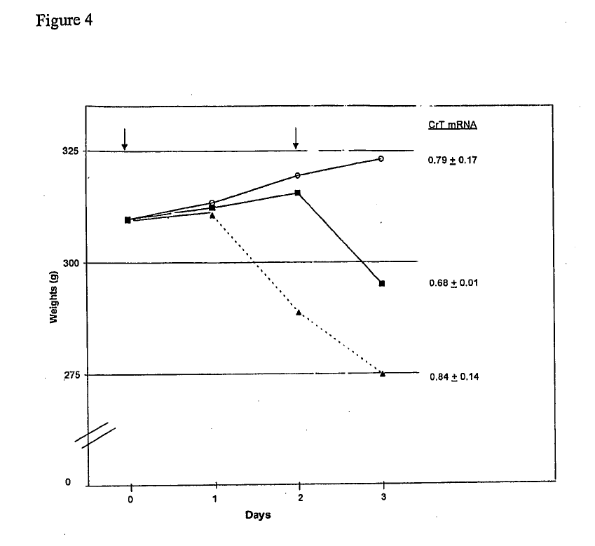

Hypothalamic mRNA concentrations for the creatine transporter (CrT) were measured using Real-Time PCR of RNA extracts from the hypothalami of beta-GPA-, vehicle- and fasted vehicle-treated rats. When normalized to amounts of L32 mRNA (a ribosomal housekeeping gene), there was a slight decrease in relative units of CrT gene expression in beta-GPA-treated compared to vehicle-treated animals, but this change did not achieve statistical significance (FIG. 4). In addition, no changes were detected in CrT gene expression in vehicle-treated rats that were fasted for 48 hours to provide a positive control for weight loss.

Protein expression (Fos) of the early immediate gene (c-fos) was examined using indirect immunofluorescence of coronal sections of the hypothalami of rats 4 hours after icv administration of beta-GPA (2 mg per rat) or vehicle. Fos IR was increased in beta-GPA-treated animals, especially around the III ventricles. Treatment with CoPP increased Fos IR from 2.79+0.98 to 4.44+2.22 percent in the paraventricular nucleus and from 2.67+0.75 to 6.86+5.85 percent in the dorsomedial nucleus. The increase in Fos IR was even more pronounced in the midsection of the ventral III ventricle where CoPP treatment increased Fos IR from 5.32+2.79 to 48.3+11.18 percent (p<0.025).

The references cited below correspond to the immediately preceding example, “example 1”.

### REFERENCES

1 Wyss M, Kaddurah-Daouk R. Creatine and creatinine metabolism, Phys. Rev. 2000; 80: 1107-1213.
2 Fitch C D, Chevil R. Inhibition of creatine and phosphocreatine accumulation in skeletal muscle and heart. Metabolism 1980; 29: 686-690.
3 Mahanna D A, Fitch C D, Fischer V W. Effects of beta-guanidinopropionic acid on murine skeletal muscle. Exp. Neurol. 1980; 68: 114-121.
4 Otten J V, Fitch C D, Wheatley J B, Fischer V W. Thyrotoxic myopathy in mice: Accntuation by a creatine transport inhibitor. Metabolism 1986; 35: 481-484.
5 Adams G R, Haddad F, Baldwin K M. Interaction of chronic creatine depletion and muscle unloading: effects on postural and locomotor muscles. Journal of Applied Physiology 1994; 77: 1198-1205.
6 Moerland T S, Kushmerick M J. Contractile economy and aerobic recovery metabolism in skeletal muscle adapted to creatine depletion. American Journal of Physiology (Cell Physiol. 36) 1994; 267: C127-C137.
7 Adams G R, Baldwin K M. Age dependence of myosin heavy chain transitions induced by creatine depletion in rat skeletal muscle. Journal of Applied Physiology 1995; 78: 368-371.
8 Boehm E, Chan S, Monfared M, Wallimann T, Clarke K, Neubauer S. Creatine transporter activity and content in the rat heart supplemented by the depleted of creatine. Amerian Journal of Physiology (Endocrinol. Metab) 2003; 284: E399-E406.
9 Wakatsuki T, Hirata F, Ohno H, Yamamoto M, Sato Y, Ohira Y. Thermogenic responses to high-energy phosphate contents and/or hindlimb suspension in rats. Japanese Journal of Physiology 1996; 46: 171-175.
10 Yamashita H, Ohira Y, Wakatsuki T, Yamamoto M, Kizaki T, Oh-ishi S, Ohno H. Increased growth of brown adipose tissue but its reduced thermogenic activity in creatine-depleted rats fed beta-guanidinopropionic acid. Biochemica et Biophysica Acta 1995; 1230: 69-73.
11 Ohira Y, Ishine S, Tabata I, Kurata H, Wakatsuki T, Sugawara S, Yasui W, Tanaka H, Kuroda Y. Non-insulin and non-exercise related increase of glucose utilization in rats and mice. Japanese Journal of Physiology 1994; 44: 391402.
12 Meglasson M D, Wilson J M, Yu J H, Robinson D D, Wyse B M, de Souza C J. Antihyperglycemic action of guanidinoalkanoic acids: 3-guanidinopropionic acid ameliorates hyperglycemia in diabetic KKAy and C57BL6Job/ob mice and increases glucose disappearance in rhesus monkeys. Journal of Pharmacology and Experimental Therapeutics 1993; 266: 1454-1462.
13 Pelouch V, Kolar F, Khuchua Z A, Elizarova G V, Milerova M, Ost'adal B, Saks V A. Cardiac phosphocreatine deficiency induced by GPA during postnatal development in rat. Mol. Cell. Biochem. 1996; 163/164: 67-76.
14 Levine S, Tikunov B, Henson D, LaManca J, Sweeney H L. Creatine depletion elicits structural, biochemical, and physiological adaptations in rat costal diaphragm. American Journal of Physiology (Cell Physiol. 40) 1996; 271: C1480-C1486.
15 Holtzman D, McFarland E, Moerland T, Koutcher J, Kushmerick M J, Neuringer L J. Brain creatine phosphate and creatine kinase in mice fed an analogue of creatine. Brain Res. 1989; 483: 68-77.
16 Holtzman D, Brown M, O'Gorman E, Allred E, Wallimann T. Brain ATP metabolism in hypoxia resistant mice fed guanidinopropionic acid. Dev Neurosci 1998; 20: 469-477.
17 Holtzman D, Meyers R, O'Gorman E, Khait I, Wallimann T, Allred E, Jensen F. In vivo brain phosphocreatine and ATP regulation in mice fed a creatine analog. Am. J. Physiol. (Cell Physiol. 41) 1997; 272: C1567-C1577.
18 Konig J F, Klippel R A. The rat brain: A stereotaxic atlas of the forebrain and lower part of the brainstem. Baltimore: Williams and Williams; 1963.
19 Galbraith R A, Kappas A. Intracerebroventricular administration of cobalt protoporphyrin elicits prolonged weight reduction in rats. Am. J. Physiol. 1991; 261: R1395-R1401.
20 Defalco A J, Davies R K. The synthesis of creatine by the brain of the intact rat. J. of Neurochem. 1961; 7: 308-312.
21 Galbraith R A, Kappas A. Regulation of food intake and body weight by cobalt porphyrins in animals. Proc. Natl. Acad. Sci. USA 1989; 86: 7653-7657.
22 Galbraith R A, Kappas A. Cobalt-protoporphyrin suppresses expression of genetic obesity in homozygous (fa/fa) Zucker rats. Pharmacology 1990; 41: 292-298.
23 Dragunow M, Faull R. The use of c-fos as a metabolic marker in neuronal pathway tracing. J. Neurosci. Methods 1989; 29: 261-265.
24 Sagar S M, Sharp F R, Curran T. Expression of c-fos protein in brain: Metabolic mapping at the cellular level. Science 1988; 240: 1328-1331.

### Example 2

### The Role of Creatine Uptake in the Brain in the Hypophagia and Weight Loss Caused by Cobalt Protoporphyrin

### Materials and Methods

Cobaltic protoporphyrin IX was purchased from Porphyrin Products (Logan, Utah). All other chemicals were of the highest reagent grade commercially available.

### Animal Handling and Treatment

All animal handling was approved by and done in accordance with the guidelines and requirements of the University of Vermont Institutional Animal Care Use Committee. Adult male Sprague-Dawley rats (mycoplasma free; 200 g) were purchased from Charles River (Quebec, Canada) and housed in an air-conditioned (23+1° C.) room with lights on for 12 hours daily (starting at 6:00 a.m.) in the University of Vermont Animal Facility. Rats were housed singly in regular cages with free access to Purina Rat Chow (RMH 3000) and water for at least five days prior to surgery. Stereotactic surgery was used to implant chronic indwelling stainless steel cannulae into the third ventricle of the brain under fentanyl/droperidol anesthesia. Coordinates for cannula placement were obtained from the Konig Klippel stereotactic surgery atlas [9]; with the nose bar 2.4 mm below the intraaural line and the vertical micrometer gantry tilted 9 degrees towards the animal, the coordinates were 4.9 mm dorsal to the intraaural line, 5 mm anterior to the intraaural line and 1.1 mm lateral to the midline [2]. A minimum of 5 days later, animals were injected icv with CoPP (which was dissolved in 0.2 N NaOH and the pH adjusted to 7.4-7.8) or other experimental compounds. Control animals received equal volumes of vehicle. Volumes of icv injections never exceeded 10 ul per animal.

In one experiment, animals were injected icv with vehicle or CoPP followed by a second immediate injection (within 5 minutes) of vehicle or creatine. The second injection regimen was repeated an additional 9 times over an 11-day period. Because of the repetitive dosing regimens, animals were trained for six days prior to the first injection to ready them for injections without anesthesia. Animals were handled daily and accustomized to several minutes of confinement in a small container sized to minimize movement, prior to being sham injected. Measures of body weight were made daily with a digital balance with a 6-second integration period to minimize the effects of animal movement.

### Differential Display

RNA Extraction from Rat Hypothalamus. Adult male Sprague-Dawley rats were randomly separated into three groups. Group 1 (control) was injected icv with saline and fed ad lib. Group 2 (fasted) was injected icv with saline and allowed ad lib access to water only; Group 3 (CoPP) was injected icv with CoPP 0.4 umol/kg body weight and fed ad lib. After 24 and 48 hours, weights were measured. Only those rats with robust weight loss of at least 10% of initial body weight 48 hours after treatment were chosen for the CoPP group. Eleven rats from each group were then killed by decapitation, using procedures outlined in the Animal Welfare Act and as approved by the University of Vermont, Office of Animal Care Management. The hypothalamic block of each rat was dissected from the rat's brain as described [10] and immediately homogenized with RNase-free homogenizer. Total RNA was extracted using an Ultraspec-II RNA kit (Biotecx Laboratories, Inc., Houston, Tex.) following the manufacturer's procedures and stored at −80 degrees C. until analysis.

Differential Display-Reverse Transcription-Polymerase Chain Reaction (DD-RT-P). The RNAimage mRNA Differential Display system was purchased from GenHunter Corporation (Nashville, Tenn.). Three anchored H-T11M (where M refer to G, A, or C) primers and 12 sets of arbitrary primers (HAP 1 to 12) were chosen to use in this particular experiment. 1 ug of RNA was added to a mixture (10 ul) containing 1 U of RQI RNase-free DNase (Promega, Madison, Wis.) and 1×RQI buffer and incubated at 37 degrees C. for 30 min. 2 ul of this mixture was further mixed with 17 ul of RT pre-mixture containing 5× buffer (4 ul), 250 uM dNTPs (1.6 ul), a 2 uM solution of one of the H-T11M primers (2 ul) and MMLV reverse transcriptase (1 ul: Invitrogen, Carlsbad, Calif.). This mixture was incubated at 60 degrees C. for 5 min, 37 degrees C. for 60 min, and 75 degrees C. for 5 min.

Thirty-two samples from the three treatment groups were set up for PCR at the same time. Master mixture was pre-made to ensure standardization of the reaction conditions. Two sets of master mixtures were used in PCR reactions; one set was made as recommended by GeneHunter and the other set was modified by us to enable amplification of longer fragments. 2 ul of cDNA from the above 20 ul of RT-reaction was mixed with 1× master mixture which consisted of 3.3×PCR buffer (6 ul), 25 mM MgACO (0.8 ul), 25 uM dNTPs (1.6 ul), rTh polymerase (0.25 ul; Perkin Elmer Applied Biosystems, Foster City, Calif.), alpha [³³P]dNTP(0.25 ul; Perkin Elmer, Boston, Mass.), the same 2 uM solution of H-T11 primer as used in the reverse transcription step (2 ul) and a 2 uM solution of one of the HAP arbitrary primers (2 ul). The reaction was run at 94 degrees C. for 5 min, then 40 cycles of 94 degrees C. for 30 seconds, 40 degrees C. for 2 min, and 72 degrees C. for 1 min. Final elongation was at 72 degrees C. for 5 min. Aliquots (3.5 ul) of each sample were mixed with 2 ul of loading dye (95% formamide, 10 mM EDTA, 0.09% xylene cyanole and bromophenol blue), denatured at 80 degrees C. for 2 min and loaded on a 6% sequencing gel which had been pre-run at 60 watts for 30 min. The gel was run at 60 watts for 8 hours for separating longer fragments, or for 5 hours for shorter fragments. After running, the gel was wrapped in plastic wrap and dried under vacuum on a gel dryer at 80 degrees C. for 1 hour. Radioactive ink was used to orientate the gel before exposing it to X-ray film.

Cloning of cDNA Bands Isolated from SSCP Gel. After exposure to film, the gels were aligned by matching the autoradiographic ink images on the film with the ink on the gels. The bands of interest were excised from the gel and placed in 1.5 ml tubes where they were soaked in ddH₂O for 10 minutes, incubated in boiling water for 15 min and centrifuged at maximum speed for 2 nin. The cDNA in the supernatant was recovered by ethanol and NaOAC precipitation and dissolved in 10 ul ddH₂O.

Aliquots of each cDNA template (4 ul) were mixed with 28 ul of pre-mixture containing 10× buffer (4 ul), 250 uM dNTPs (4.2 ul), [alpha-³³P] dATP (0.4 ul), and Gold Taq (0.4 ul; Perkin Elmer Applied Biosystems), together with 4 ul of each related HT11M primer and HAPM primer in a final reaction volume of 40 ul. The re-amplification was run with the same PCR regimen as described for DD-PCR except that the first step at 94 degrees C. was extended to 14 min to activate Gold Taq polymerase. PCR product (2 ul) was added to 10 ul loading buffer (0.1% SDS, 10 mM EDTA) and 6 ul of the mixture denatured with an equal volume of loading buffer in boiling water for 5 min prior to quickly chilling on ice and loading onto a 10% non-denaturing acrylamide gel (SSCP gel). The gel was run at 8 watts/750 v for 8-9 hours, then dried prior to autoradiography. The isolated single SSCP band was excised and re-amplified following the same procedures described above. The PCR products of re-amplification were directly cloned into the GenHunter PCR-TRAP Cloning System following the manufacturers' suggested procedures. Briefly, PCR product (5 ul) was added to ligation mixture (15 ul) containing 5× ligation buffer (4 ul), insert-ready PCR-TRAP Vector (2 ul) and T4 DNA ligase (GIBco; 1 ul) and incubated overnight at 16 degrees C. The second day, 10 ul of each ligation mixture was added to 100 ul of thawed GH-component cells, mixed well and incubated on ice for 45 min. The cells were heat-shocked for 2 min at 43 degrees C. and set back on ice for another 2 min; then the cells were mixed with 400 ul LB medium and incubated at 37 degrees C. for 1 hour. After vortexing briefly, 200 ul of cells from each ligation were plated, in triplicate, onto pre-warmed and numbered LB-tetracycline plates and incubated at 37 degrees C. overnight. The colonies with insert were recognized by PCR amplification with related primers, and these were grown in new LB medium prior to storage.

### Northern Blot

Northern Blots were used to confirm the results we obtained from the differential display. Six additional rats from each group (saline, CoPP, and fasted) were used in Northern Blot experiments. Total RNA (15 ug) extracted from the hypothalami of these rats was loaded on 1.3% formaldehyde-agarose gels. After 3.5 hours running at 56V, the gels were transferred to Hybrond-N+ membrane (Amersham, Piscataway, N.J.). The cloning inserts were released from PCR-TRAP Vectors by PCR amplification and then gel-purified with the Wizard®DNA Clean-up System (Promega). The inserts were labeled with [alpha³²P]dCTP (Perkin Elmer) using a random primers DNA labeling kit (Invitrogen, San Diego, Calif.). Labeled probe (25 ng) was denatured and then hybridized with a membrane which had been pre-incubated for 15 min in 15 ml Rapidhyb buffer (Amersham) in a Roller-Blot Hybridizer (Techne HB-3D, Cambridge, UK) for 1 hour. After washing under different stringencies, depending on the probes used, the membranes were exposed overnight to film. The autoradiographs were scanned using a GS-700 imaging densitometer (BioRad, Inc., Hercules, Calif.) and analyzed using Quantity One software (BioRad). A series of primer sets were designed using available sequences for inserts or for related mRNA cds.

### Real-Time PCR

RNA was purified with RNAase-free DNase (Promega) at 37 degrees C. for 30 min. The first strand of cDNA was synthesized by adding 1 ug of purified RNA from each sample to 20 ul containing MMLV reverse transcriptase (Invitrogen Life Tech). Creatine transporter (CrT) gene expression was further quantatified by Real-Time PCR using sense primer: 5′GGTAAGGGTGCAAGCCTTTG′ (SEQ ID NO: 1); antisense primer: 5′TTGCTATGTTTGCAGTGGCT-3′ (SEQ ID NO:2) and TaqMan probe: 5′ACATTCTTACTGTGCTAAAAAAGCCACTGC-3′ (SEQ ID NO: 3) (MMG-Biotech). The rat ribosomal protein L32 gene was used as a loading control. Each cDNA sample (1 ul) derived from the 20 ul reverse transcription reaction and 1 ul RNA template was mixed with 24 ul master mixture (Perkin Elmer) in a 96-well TaqMan plate. The PCR was run in an ABI prism 7700 thermocycler (Applied Biosystems) at 50 degrees C. for 2 min and 95 degrees C. for 10 min; this was followed by 95 degrees C. for 15 sec and 60 degrees C. for 1 min for 40 cycles. The data were analyzed using software supplied with the ABI prism 7700.

### In Situ Hybridization

At least 3 rats per group were used for each individual probe. Brains were dissected as described above and were cut vertically to remove the anterior portion at the optic chiasm and the posterior portion at the interpeduncular fossa so that the remaining blocks included the whole hypothalamus with a small additional margin of adjacent tissue. The blocks were immediately frozen on foil on dry ice, then stored at −80 degrees C. Blocks were sectioned in a cryostat (Micron HM500O) at −20 degrees C. and at a thickness of 13 microns. The slides were fixed in 4% paraformaldehyde for 5 minutes and acetylated in 1.3% triethanolamine and 0.5% acetic anhydride, pH 8.0 for 5 minutes. Sections were then dehydrated sequentially in 70%, 95% and 100% ethanol. After air drying, the slides were ready to use.

For preparation of radiolabeled riboprobes, the DNA fragments containing the creatine transporter insert and a T7 or SP6 phage promoter sequence in each side were produced from cloning vectors (pCRII-TOPO, Invitrogene; PCR-TRAP, GenHunter) either by restriction endonuclease digestion or by PCR amplification. The fragments were purified utilizing the Wizard®DNA Clean-up System (Promega). The antisense or sense riboprobes were synthesized and labeled in an in vitro transcription mixture that consisted of 1 ul 10× buffer, 0.5 ul each of 10 mM solutions of rATP, rGTP, and rUTP, 0.6 ul 0.2 mM rCTP, 20 ul Rnasin RNase inhibitor (Promega), 1.5 ug DNA template, 3 ul [³⁵S]-5′alpha-CTP, and 50 ul T7 or SP6 polymerase (Biolabs Inc., Beverly, Mass.) at 37 degrees C. for 1 hour. The probes then underwent DNase digestion to remove remaining template and a phenol-chloroform purification to remove DNase. The cpm value was determined by counting in a liquid scintillation spectrometer.

The slides were incubated in pre-hybridization solution (2×SSC, 50% formamide) at 50 degrees C. for 1 hour, and then 10⁶ cpm of denatured riboprobe mixed with 50 ul of hybridization solution (25% 50×Denhardt's, 50% deionized formamide, 10% dextran sulfate, 100 mM Tris, 1 mM EDTA, 1M NaCl, 0.5 mg/ml tRNA, 0.1 mg/ml ssDNA) was loaded onto each slide and sealed with coverslips. The slides were incubated in a humid chamber at 50 degrees C. overnight. The second day, after soaking off the coverslips, the slides were washed with RNase solution (RNase A 2 ug/ml at 37 degrees C. for 30 minutes); washing was repeated with different stringencies. Tissues were then dehydrated in increasing concentrations of ethanol, the slides air-dried, and exposed to film for 7 days.

For dipping and developing in situ slides, Kodak NTB-2 emulsion was diluted (1:1) using ddH₂O and warmed to 40 degrees C. in a dark room. The slides were dipped in emulsion and the backs of the slides were wiped clean, followed by air-drying in the dark for at least 1 hour. The emulsion was then exposed by storing the slides in the dark at 4 degrees C. for approximately 3 weeks. After autoradiography, the slides were developed in Kodak D19 developer for 5 minutes then fixed in Kodak Fixer for 4 minutes then, in turn, passed through a series of washing and dehydrating steps. After incubating in xylene for 5 minutes, the slides were mounted with Permount and coverslipped. Slides were observed under dark-field microscopy (Olympus, Tokyo, Japan) and silver grains in regions of interest were assessed. Images were captured at 4× magnification and quantitated utilizing the Metamorph system and software. Areas of interest were outlined using the system's electronic pen as either boxes (for the third ventricle and dorsomedial nucleus) or circles/elipses (for the paraventricular, arcuate, ventromedial and habenular areas). Hybridization in the specific areas of interest were then quantified, using the Hue Saturation Intensity modality, by expressing the summated area of the silver grains as percentages of the total area outlined by the electronic pen. Areas of interest were quantitated on each section in which they appeared and averaged to yield means+SEM of percentages. Bilateral structures were counted separately and averaged to yield means+SEM of percentages.

### Immunohistochemistry Experiments

In experiments involving immunohistochemistry, animals were injected icv with CoPP 0.4 umol/kg body weight or with vehicle. Four hours after injection, animals were anesthetized with halothane and perfused through the left ventricle of the heart, initially with oxygenated Krebs buffer and then with ˜350 ml of 4% paraformaldehyde in 0.1M NaCl, pH 7.4 phosphate buffered saline (PBS). Brains were extracted from the skull, hypothalamic blocks dissected (by coronal cuts anterior and posterior to hypothalamic landmarks) and post-fixed in 4% paraformaldehyde solution at 4 degrees C. overnight. The following day, the blocks were transferred to 30% sucrose and incubated overnight at 4 degrees C. After freezing in OCT (Sakura, Japan), blocks were sectioned coronally at 40 um on a cryostat and sections individually floated in multi-well dishes filled with PBS. Sections were treated for 30 minutes with 0.4% Triton detergent in potassium PBS to aid in antibody penetration. Primary antibodies were added, the plates shaken gently for 1 hour at room temperature, then incubated at 4 degrees C. for 72 hours. Sections were then brought to room temperature while shaking and the primary antibody solution was removed by washing 3 times with PBS buffer. After removal of the last wash, the secondary antibody solution was added and the plates incubated for 2 hours at room temperature in the dark. Removal of the second antibody was achieved by washing three times with PBS buffer prior to mounting on subbed slides. They were allowed to dry, rewetted with PBS buffer and cover-slipped with Citifluor (Ted Pella, Reading, Calif.).

Creatine transporter (CrT) and Fos studies were performed using indirect fluorescence visualization with rabbit anti-CrT (1:40; Alpha Diagnostics, San Antonio, Tex.) with goat anti-rabbit Cy3 (Jackson Immuno Research Laboratories, Inc., West Grove, Pa.) and rabbit c-Fos-AB5 (1:2,000; Oncogene Research Products, Cambridge, Mass.) with goat anti-rabbit Cy3 (1:500; Jackson Immuno Research Laboratories, Inc.) as the secondary antibody. Sections were examined using an Olympus fluorescence microscope and digital images captured with MagnaFire 2.1 Software and imported into Metamorph 6.1 for quantitation of fluorescence. Tissue incubated in the absence of primary or secondary antisera showed no reaction products. Staining observed in experimental tissue was compared to that observed from experiment-matched negative controls. Areas of interest were outlined with an electronic pen and the intensity of fluorescence was expressed as a percentage of the total area of the region of interest. Areas of interest were quantitated on each section in which they appeared and averaged to yield means+SEM of percentages. Bilateral structures were counted separately and averaged to yield mean+SEM percentages.

### Creatine Assay

Hypothalamic blocks were rapidly dissected from animals treated icv with vehicle or CoPP at the indicated doses and snap-frozen in liquid nitrogen. They were stored at −80 degrees C. until they were crushed into a powder under liquid nitrogen and homogenized in 0.5 M PCA. Samples were then centrifuged at 4 degrees C. for 10 minutes at 15,000×g and the resulting supernatant neutralized with 1 M KOH. HPLC analysis was performed essentially as described [11]. Samples were injected onto a Partisil 10 SAX analytical column which was eluted linealy starting with Buffer A (0.01M H₂PO₄) and changing to Buffer B (0.75M KH₂PO₃). The column was run on a Waters HPLC equipped with a Waters 996 Photodiode Array Detector and a Waters 717+ Auto Sampler. Both creatine and phosphocreatine standards were run with each sample set. Results are expressed as nanomoles per mg wet weight.

### Statistics

Results from Northern blot densitometry or Real-Time PCR for creatine transporter mRNA were normalized to L32 ribosomal protein mRNA concentrations and expressed in arbitrary units as mean+SEM, and were analyzed by single factor ANOVA. Analysis of mean percentage staining for CrT in situ hybridization and CrT immunoreactivity was performed non-parametrically using the Kruskal-Wallis Test. Creatine and phosphocreatine concentrations in the hypothalamus were expressed as nmol/mg wet weight (means+SEM) and were analyzed by ANOVA.

### Results

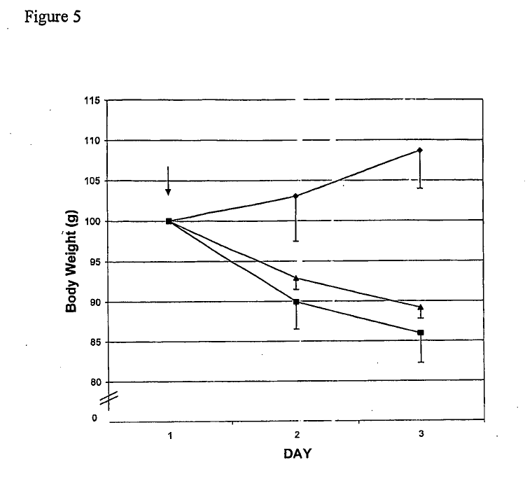

Adult male Sprague-Dawley rats were used in the differential display experiment. Rats were selected to form three groups; vehicle-treated (n=11), CoPP-treated (n=11) and vehicle-treated and fasted (n=10). Only animals which lost a minimum of 10% of body weight 48 hours after icv treatment with CoPP 0.4 um/kg body weight were included in the CoPP-treated group. Vehicle-treated fasted animals lost between 6% and 20% of body weight during this same 48-hour period. The mean percentage changes in weights of the animals used are displayed in FIG. 5.

The differential display strategy used (two sets of 8 primers each) theoretically displays cDNAs from approximately 65% of the total number of mRNA species present in the sample. One of the cDNA's amplified from hypothalamic mRNAs, arbitrarily designated as11G2L, was found to be absent in all samples from both fed and fasted rats treated icv with vehicle, but present in those from CoPP-treated rats. These bands, in addition to some negative controls, were excised from the gels, the cDNAs recovered and re-amplified (as in the differential display) and the products separated on an SSCP gel. This procedure yielded a single band which was excised and re-amplified again prior to direct insertion into the Gene Hunter PCR-TRAP Cloning System.

The fidelity of these procedures was confirmed by labeling the cloned insert and using it as the probe in Northern blots with freshly isolated total RNA from hypothalami harvested from additional rats (saline, CoPP or fasted). Densitometry of the 11G2L bands, normalized to that of the housekeeping gene L32, revealed that 11G2L expression from CoPP-treated animals was increased significantly (p<0.02) by approximately 20% compared to that from vehicle-treated animals, either fed or fasted (Table 1). A similar significant (p<0.02) result was obtained from Real-Time PCR carried out on the same samples (Table 1).

TABLE 1
Confirmation of the effect of icv CoPP administration
on mRNA concentrations of the band identified
by differential display as 11G2L.

| | Δ Weight | Northern Blot | |
|---|---|---|---|
| | % | Densitometry | Real-Time PCR |
| Vehicle | +4.3 | 0.85± | 0.87 ± 0.059 |
| CoPP | −29 | 1.023±* | 0.97 ± 0.050* |
| Fasted | −12 | 0.79± | 0.84 ± 0.060 |

The band 11G2L isolated from the differential display (see FIG. 5) was re-amplified and cloned as described in Methods. Cloned inserts were released, amplified and labeled prior to use in Northern hybridization studies. Total mRNA was prepared from newly-treated rats. The mean weight changes of each group of rats (vehicle-treated, CoPP-treated and vehicle-treated fasted, 6 animals per group), normalized as weight at icv injection divided by weight at sacrifice 48 hours later, are presented as Δ weight (%). Northern blots were scanned and densitometry readings normalized to L32 ribosomal protein mRNA concentrations to yield arbitrary units which are presented as means ± SEM. Real-Time PCR was also performed (as described in Methods) in these newly prepared RNA samples. Results are expressed as arbitrary units after normalization to L32 ribosomal protein mRNA concentrations (means ± SEM).
*= p < 0.02 compared to vehicle controls (by single factor ANOVA).

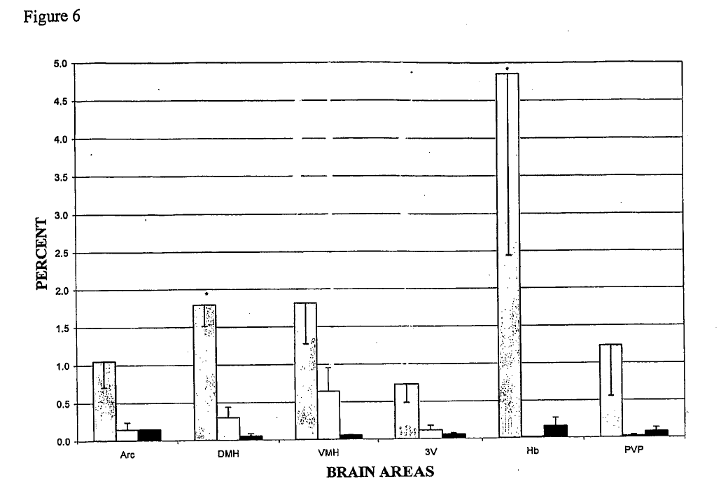

The cloned inserts were sequenced and Genbank searched using the BLAST system for matches. The cloned sequence (106 bp) was found to be 100% homologous for 77 bp with NM 017348.1 Rattus Norvegicus Choline Transporter (CHOT1) mRNA (3972 bp). It was also homologus for 59 of 64 bp with L31409.1 Homo Sapiens Creatine Transporter mRNA (3747 bp). This gene, identified by Mayser et. al. [12] was originally considered to encode a choline transporter but has subsequently been shown to encode a creatine transporter [13; 14] which is widely distributed in tissues of animals and man [15]. To examine the distribution of the increase in CrT mRNA after treatment with CoPP, we performed in situ hybridization studies using radioactively labeled probes to detect CrT mRNA in the brains of rats 48 hours after icv treatment with vehicle or CoPP 0.4 um/kg body weight. Treatment with CoPP was associated with an increase in the frequency of silver grains observed with darkfield microscopy of radioautography emulsion-dipped coronal sections. Hybridization was increased after CoPP compared to control treatment (as a percentage of the summated area occupied by silver grains compared to the total area of the specific brain nucleus or structure) in the arcuate, dorsomedial, ventromedial and third ventricle areas of the hypothalamus and in the paraventricular thalamic and habenular areas (see FIG. 6). Animals which had been fasted for 48 hours acted as positive controls for weight loss; the lack of an increase in CrT in situ hybridization in these controls (FIG. 6) indicates that the increase in CrT expression in CoPP-treated animals does not arise simply as a compensatory result of weight loss.

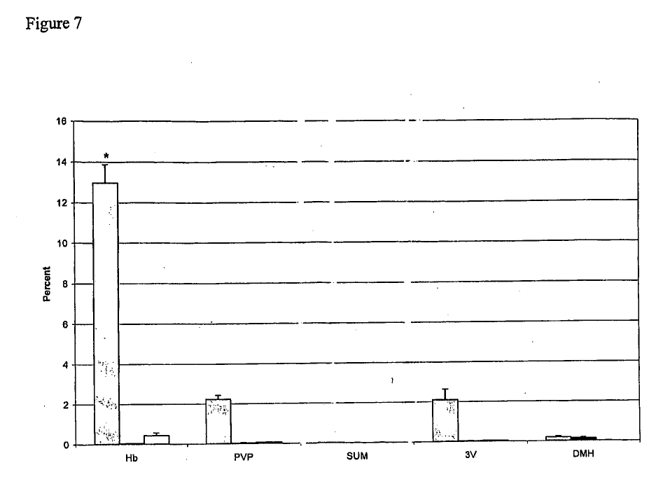

Further studies were carried out to determine if the change in expression of the CrT gene with CoPP treatment also resulted in alterations in the amounts of immunoreactive CrT protein. Indirect immunofluorescence studies were performed on sections from rats 4 hours after icv treatment with vehicle or CoPP 0.4 um/kg body weight. As shown in FIG. 7, immunoreactive (IR) CrT was increased in the dorsomedial nucleus, the supramamillary nucleus, around the III ventricle and extrahypothalamically in the habenular (p<0.05) and paraventricular thalamic areas in CoPP-treated animals compared to controls and fasted controls.

In order to determine whether or not these changes in CrT mRNA and protein immunoreactivity were paralleled by changes in creatine concentration, hypothalamic concentrations of creatine and phosphocreatine were measured by HPLC. Results are shown in Table 2. Treatment with CoPP 0.4 um/kg body weight 48-hours earlier resulted in a significant decrease (p=0.008) of about 20% in the concentrations of creatine in hypothalamic blocks from male rats compared to vehicle-treated or fasted rats. There was an accompanying decrease of about 40% in phosphocreatine concentrations which failed to meet statistical significance.

TABLE 2
The Effect of Treatment with CoPP on Hypothalamic
Creatine and Phosphocreatine Concentrations

| | Vehicle | CoPP | Fasted |
|---|---|---|---|
| Creatine | 12.32 + 0.79 | 9.80* + 0.50 | 10.28 + 0.68 |
| (nmol/mg wet wt) | (n = 15) | (n = 20) | (n = 14) |
| Phosphocreatine | 0.47 + 0.10 | 0.27** + 0.05 | 0.39 + 0.07 |
| (nmol/mg wet wt) | (n = 8) | (n = 10) | (n = 8) |

Three separate groups of male rats were treated icv with CoPP 0.4 umol/kg b.w., vehicle or vehicle and fasted. Forty eight hours later, all animals were killed and hypothalamic blocks dissected and frozen in liquid nitrogen. After homogenization and processing as described in Methods, HPLC was used to measure creatine and phosphocreatine (except that the latter was measured on only a single group of animals). Means + SEM of the indicated number of animals are presented.
*indicates p = 0.008 and
**indicates p = 0.072, both versus vehicle-treated controls (ANOVA).

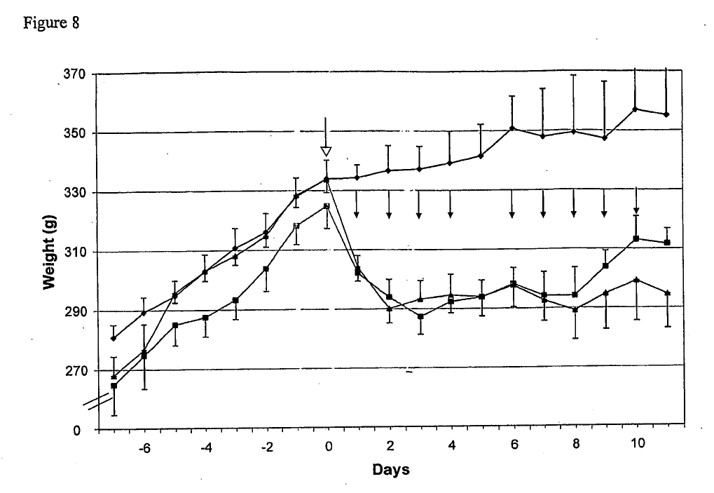

In light of the decrease in hypothalamic creatine concentrations following treatment with CoPP, experiments were conducted to determine if the extent or course of weight loss after CoPP could be modified by icv administration of creatine. As shown in FIG. 8, repetitive dosing of both CoPP-treated and vehicle-treated animals with 10 icv dosages of 0.5 mg of creatine was without effect on their course of weight gain (controls) or loss (CoPP-treated).

The references cited below correspond to the immediately preceding example, “example 2”.

### REFERENCES

1 Galbraith R A, Kappas A. Regulation of food intake and body weight by cobalt porphyrins in animals. Proc. Natl. Acad. Sci. USA 1989; 86: 7653-7657.
2 Galbraith R A, Kappas A. Intracerebroventricular administration of cobalt protoporphyrin elicits prolonged weight reduction in rats. Am. J. Physiol. 1991; 261: R1395-R1401.
3 Galbraith R A, Kappas A. The effects of cobalt-protoporphyrin on food intake, body weight and metabolism in adult male rats. Am. J. Physiol. 1991; 261: R1388-R1394.
4. Galbraith R A, Kappas A. Cobalt protoporphyrin regulates body weight in beagle dogs: Induction of weight loss in normal animals of stable adult weight. Pharmacology 1991; 43: 96-105.
5. Galbraith R A, Kappas A. Cobalt-protoporphyrin suppresses expression of genetic obesity in homozygous (fa/fa) Zucker rats. Pharmacology 1990; 41: 292-298.
6. Galbraith R A, Kow L M, Pfaff D, Kappas A. Injection of cobalt protoporphyrin into the medial nuclei of the hypothalamus elicits weight loss. Am. J. Physiol. 1992; 263: R805-R812.
7. Ghezzi P, Saccardo B, Villa P, Rossi V, Bianchi M, Dinarello C A. Role of interleukin-1 in the depression of liver drug metabolism by endotoxin. Infect. Immun. 1986; 54: 837-840.
8. Turner M B, Corp E S, Galbraith R A. Lack of NPY-induced feeding in cobalt protoporphyrin-treated rats is a postreceptor defect. Physiol. Behav. 1994; 56: 1009-1014.
9. Konig J F, Klippel R A. The rat brain: A stereotaxic atlas of the forebrain and lower part of the brainstem. Baltimore: Williams and Williams; 1963.
10. Glowinski J, Iversen L L. Regional studies of catecholamines in the rat brain. J. Neurochem. 1966; 13: 655-669.
11. Carter A J, Mula R E. Application and validation of an ionic xchange high performance liquid chromatographic method for measuring adenine nucleotides, creatine and creatine phosphate in mouse brain. J. Chromatogr. 1990; 527: 31-39.
12. Mayser W, Schloss P, Betz H. Primary structure and functional expression of a choline transporter expressed in the rat nervous system. FEBS Lett. 1992; 305: 31-36.
13. Schloss P, Mayser W, Betz H. The putative rat choline transporter CHOT1 transports creatine and is highly expressed in neural and muscle-rich tissues. Biochem. Biophys. Res. Comm. 1994; 198: 637-645.
14. Gonzalez A M, Uhl G R. ‘Choline/orphan V8-2-1/creatine transporter’ mRNA is expressed in nervous, renal and gastrointestinal systems. Mol. Brain. Res. 1994; 23: 266-270.
15. Wyss M, Kaddurah-Daouk R. Creatine and creatinine metabolism. Phys. Rev. 2000; 80: 1107-1213.
16. Stockler S, Holzbach U, Hanefeld F, Marquardt I, Helms G, Requart M, Hanicke W, Frahm J. Creatine deficiency in the brain: A new, treatable inborn error of metabolism. Pediatr. Res. 1994; 36: 409-413.
17. Bianchi M C, Tosetti M, Fornai F, Alessandri M G, Cipriani P, De Vito G, Canapicchi R. Reversible brain creatine deficiency in two sisters with normal blood creatine level. Ann. Neurol. 2000; 47: 511-513.
18. Hahn K A, Salomons G S, Tackels-Horne D, Wood T C, Taylor H A, Schroer R J, Lubs H A, Jakobs C, Olson R L, Holden K R, Stevenson R E, Schwartz C E. X-linked mental retardation with seizures and carrier manifestations is caused by a mutation in the creatine-transporter gene (SLC6A8) located in Xq28. Am. J. Hum. Genet. 2002; 70: 1349-1356.
19. Leuzzi V, Bianchi M C, Tosetti M, Carducci C, Cerquiglini A, Cioni G, Antonozzi I. Brain creatine depletion: Guanidinoacetate methyltransferase deficiency (improving with creatine supplementation). Neurology 2000; 55: 1407-1409.
20. Dedeoglu A, Kubilus J K, Yang L, Ferrante K L, Hersch S M, Beal M F, Ferrante R J. Creatine therapy provides neuroprotection after onset of clinical symptoms in Huntington's disease transgenic mice. J. Neurochem. 2003; 85: 1359-1367.
21. Defalco A J, Davies R K. The synthesis of creatine by the brain of the intact rat. J. of Neurochem. 1961; 7: 308-312.
22. Tarnopolsky M A. Potential benefits of creatine monohydrate supplementation in the elderly. Current Opinion in Clinical Nutrition and Metabolic Care 2000; 3: 497-502.
23. Braissant O, Henry H, Loup M, Eilers B, Bachmann C. Endogenous synthesis and transport of creatine in the rat brain: an in situ hybridization study. Mol. Brain. Res. 2001; 86: 193-201.
24. Moller A, Hamprecht B. Creatine transport in cultured cells of rat and mouse brain, J. Neurochem. 1989; 52: 544-550.
25. Happe H K, Murrin L C. In situ hybridization analysis of CHOT1, a creatine transporter, in the rat central nervous system. J. Comp. Neurol. 1995; 351: 94-103.
26. Wang W, Shang L-H, Jacobs D O. Complement regulatory protein CD59 involves c-SRC related tyrosine phosphorylation of the creatine transporter in skeletal muscle during sepsis. Surgery 2002; 132: 334-340.
27. Zhao C-R, Shang L, Wang W, Jacobs D O. Myocellular creatine and creatine transporter serine phosphorylation after starvation. J. Surg. Research 2002; 105: 10-16.
28. Loike J D, Zalusky D L, Kaback E, Miranda A F, Silverstein S C. Extracellular creatine regulates creatine transport in rat and human muscle cells. Proc. Natl. Acad. Sci. U.S.A 1988; 85: 807-811.
29. Guerrero-Ontiveros M L, Wallimann T. Creatine supplementation in health and disease. Effects of chronic creatine ingestion in vivo: down-regulation of the expression of creatine transporter isoforms in skeletal muscle. Mol. and Cell. Biochem. 1998; 184: 427-437.
30. Tarnopolsky M, Parise G, Fu M H, Brose A, Parshad A, Speer O, Wallimann T. Acute and moderate-term creatine monohydrate supplementation does not affect creatine transporter mRNA or protein content in either young or elderly humans. Mol. and Cell. Biochem. 2003; 244: 159-166.
31. Tran T T, Dai W, Sarmalkar M. Cyclosporin A inhibits creatine uptake by altering surface expression of the creatine transporter. J. Biol. Chem. 2000; 275: 35708-35714.
32. Rebaudo R, Melani R, Carita F, Rosi L, Piccio V, Ruggeri P, Izvarina N, Balestrino M. Increase of cerebral phosphocreatine in normal rats after intracerebroventricular administration of creatine. Neurochem. Res. 2000; 25: 1493-1495.

1. A method of promoting weight loss, or treating or preventing a body disorder related to excess weight, in a subject, comprising administering intracranially to the subject an effective amount of a creatine uptake inhibitor.

2. The method of claim 1, wherein the creatine uptake inhibitor is a creatine analog.

3. The method of claim 1, wherein the creatine uptake inhibitor is a creatine phosphate analog.

4. The method of claim 1, wherein the creatine analog is beta-GPA.

5. The method of claim 1, wherein the creatine uptake inhibitor is a creatine transporter modulator.

6. The method of claim 1, wherein the creatine uptake inhibitor is administered along with a pharmaceutically acceptable carrier.

7. The method of claim 1, wherein the creatine uptake inhibitor is targeted to the brain.

8. The method of claim 7, wherein the creatine uptake inhibitor is conjugated to a brain targeting moiety.

9. The method of claim 8, wherein the creatine uptake inhibitor is conjugated to a hypothalamic targeting moiety.

10. The method of claim 1, wherein the intracranial administration is direct administration to the brain.

11. The method of claim 1, wherein the intracranial administration is direct administration to the hypothalamus.

12. The method of claim 1, wherein the intracranial administration is direct administration to the third ventricle of the brain.

13. The method of claim 1, wherein the intracranial administration is indirect.

14. The method of claim 13, wherein the indirect intracranial administration is via enteral administration of a creatine uptake inhibitor targeted to the brain.

15. The method of claim 13, wherein the indirect intracranial administration is via parenteral administration of a creatine uptake inhibitor targeted to the brain.

16. The method of claim 1, wherein the creatine uptake inhibitor is administered in combination with standard therapies used to promote weight loss or treat or prevent a body disorder related to excess weight.

17. A method of screening for a novel compound, comprising administering a test compound to a cell and measuring creatine transporter expression.

18. The method of claim 17, wherein the cell expresses creatine transporter.

19. The method of claim 17, wherein the cell is a mammalian cell.

20. The method of claim 17, wherein the cell is a human cell.

21. The method of claim 19, Wherein the cell is in a mammalian host.

22. The method of claim 21, wherein the mammalian host is a mouse or a rat.

23. (canceled)

24. (canceled)

25. (canceled)

26. (canceled)

27. (canceled)

28. (canceled)

29. (canceled)


SEQUENCE LISTING (3 sequences)

SEQ ID NO: 1 - DNA, 20 bp, Artificial Sequence
  Description of Artificial Sequence Synthetic primer
ggtaagggtgcaagcctttg

SEQ ID NO: 2 - DNA, 20 bp, Artificial Sequence
  Description of Artificial Sequence Synthetic primer
ttgctatgtttgcagtggct

SEQ ID NO: 3 - DNA, 30 bp, Artificial Sequence
  Description of Artificial Sequence Synthetic probe
acattcttactgtgctaaaaaagccactgc